(19) United States
(12) Patent Application Publication
(10) Pub. No.: US 2015/0290344 A1
(43) Pub. Date: Oct. 15, 2015

(54) BIODEGRADABLE POLYESTER- AND POLY(ESTER AMIDE) BASED X-RAY IMAGING AGENTS

(71) Applicants: Clemson University, Clemson, SC (US); College of Charleston, Charleston, GA (US)

(72) Inventors: Frank Alexis, Greenville, SC (US); Daniel C. Whitehead, Clemson, SC (US); Brooke A. Van Horn, Charleston, SC (US)

(21) Appl. No.: 14/683,775

(22) Filed: Apr. 10, 2015

Related U.S. Application Data
(60) Provisional application No. 61/978,535, filed on Apr. 11, 2014.

(51) Int. Cl. A61K 49/04; C08L 67/04; A61L 31/18; A61L 17/14; A61L 29/08; A61L 29/18; A61L 27/50; A61L 27/34; C08G 63/91; A61L 31/10

(52) U.S. Cl. CPC A61K 49/0442; C08G 63/91; C08G 63/912; C08L 67/04; A61L 31/18; A61L 31/10; A61L 29/085; A61L 29/18; A61L 27/50; A61L 27/34; A61L 17/145

(57) ABSTRACT

Biodegradable, radio-opaque polyesters and poly(ester amides) are described herein. The polyesters contain a plurality of radio-opaque agents or radio-opaque agent-containing moieties that are covalently bound along or from the polymer backbone. The agents/moieties may be bound to the termini of the polymer provided they are bound within the polyester backbone as well. The polyester can be aliphatic or aromatic. The polyester and poly(ester amide) is substituted with a plurality of radio-opaque graft agents or prepared from an appropriate radio-opaque monomer agent. The materials can be used for any application where a radio-opaque material is desired or necessary. The materials can be used to form, in whole or in part, a medical device, or coating thereon or therein.

### CROSS-REFERENCE TO RELATED APPLICATIONS

This application claims priority to U.S. Ser. No. 61/978,535, filed on Apr. 11, 2014, which is incorporated herein in its entirety.

### FIELD OF THE INVENTION

This invention is in the field of radio-opaque material, particularly biodegradable, radio-opaque polymeric materials, such as polyesters.

### BACKGROUND OF THE INVENTION

Biomedical imaging technologies can be used for both diagnostic and therapeutic purposes, thus making imaging science a critical part of the success of a patient treatment plan in a clinical setting. The technologies most commonly used are generally either non- or minimally invasive and include imaging modalities such as magnetic resonance imaging (MRI), ultrasound, optical imaging (such as near infrared and fluorescence), positron emission tomography (PET), and X-ray/computed tomography (CT) imaging.

X-ray and CT are: (1) non-invasive; (2) relatively inexpensive; (3) and broadly available to patients. Most of the currently utilized X-ray and CT imaging agents are small molecules with covalently bound iodine that allow for high X-ray attenuation but only when the contrast molecule is in locally high concentrations. These small molecules suffer from non-specific and not easily defined residence in the blood pool and tissues, and experience rapid clearance from circulation by the kidneys and liver. Additionally, they often have to be administered in high doses to produce significant imaging capability. Such high dosages, however, can result in adverse side effects.

There are many parallel strategies under investigation to address the challenge of preparing well-defined X-ray opaque materials that have controllable and/or predictable biodistribution. Examples include “packaging” of the contrast agent within stabilized organic structures including conventional liposomes, micelles, and emulsions. Unfortunately, these methods of imparting contrast to the material can still suffer from the “leakage” of the contrast agent from the material over time.

Other polymeric structures and architectures such as dendrimers, linear, block, graft, and hyperbranched polymers functionalized at the end group(s) have also being investigated. Unfortunately, these methods of imparting contrast to the material can still suffer from low imaging contrast properties because of the limited end groups available. Increased molecular weight is expected to significantly decrease the imaging contrast properties. Other strategies have focused on the covalent attachment of iodine or iodine-containing molecules to the polymer chains, particles or matrices. However, there are limited reports of fully biocompatible and biodegradable materials with sufficient X-ray opacity to meet the clinical needs of the imaging community, for example, due to limited ability to covalently attach the radio-opaque agent to the polymer (e.g., attached to the termini only).

There exists a need for biocompatible and biodegradable materials with sufficient X-ray opacity for clinical applications.

Therefore, it is an object of the invention to provide biocompatible and biodegradable materials with sufficient X-ray opacity for clinical applications.

### SUMMARY OF THE INVENTION

Biodegradable, radio-opaque polyesters and poly(ester amides) (PEAs) are described herein. In some embodiments, the polyesters and PEAs are also biocompatible. The polyesters and PEAs contain a plurality of radio-opaque agents or radio-opaque agent-containing moieties that are covalently bound along or from the polymer backbone. The agents/moieties may be bound to the termini of the polymer provided they are bound within the polyester or poly(ester amide) backbone as well. The polyester or PEA can be aliphatic, aromatic, or combinations thereof. The aliphatic and/or aromatic polyester or PEA can also include saturated and/or unsaturated groups on the backbone and/or side chains. The polyester or PEA can contain one (e.g., homopolymer), two (e.g., copolymer), three (e.g., terpolymer) or more different monomer units. In addition, the monomers in the polyester or PEA can be arranged randomly, in blocks or in alternating order. The polyester or PEA can be amphiphilic, hydrophilic or hydrophobic. The polyester or PEA can be positively charged, negatively charged or neutral.

In some embodiments, the polyesters or PEAs described herein are linear, branched, star-shaped, brush-shaped, comb-shaped, ladder-shaped, hyperbranched, dendrimeric polymers, or combinations thereof.

In some embodiments, the polyester is cross-linked or inter-linked with a second polyester that contains or lacks a radio opaque agent. In some embodiments, the polyester is cross-linked or inter-linked with a second polymer that is hydrophilic, hydrophobic or amphiphilic. In some embodiments, the polyester is cross-linked or inter-linked with small molecules. In some embodiments, the polyester is mixed with a second polymer that is hydrophilic, hydrophobic or amphiphilic. In some embodiments, the hydrophilic polymer in the co-polymer or mixture is polyethylene glycol (PEG). In some embodiments, the hydrophobic polymer in the co-polymer or mixture is poly-lactic acid (PLA) in the D- or L-isomer, or both D- and L-isomers are present in the hydrophobic polymer. In some embodiments, the amphiphilic polymer in the co-polymer or mixture is PLA-PEG.

In some embodiments, the PEA is cross-linked or inter-linked with a second PEA that contains or lacks a radio opaque agent. In some embodiments, the PEA is cross-linked or inter-linked with a second polymer that is hydrophilic, hydrophobic or amphiphilic. In some embodiments, the PEA is cross-linked or inter-linked with small molecules. In some embodiments, the PEA is mixed with a second polymer that is hydrophilic, hydrophobic or amphiphilic. In some embodiments, the hydrophilic polymer in the co-polymer or mixture is polyethylene glycol (PEG). In some embodiments, the hydrophobic polymer in the co-polymer or mixture is poly-lactic acid (PLA) in the D- or L-isomer, or both D- and L-isomers are present in the hydrophobic polymer. In some embodiments, the amphiphilic polymer in the co-polymer or mixture is PLA-PEG.

The polyesters can be prepared by the reaction of one or more hydroxy acids (e.g., glycolide, lactide, caprolactone) or by ring-opening polymerization (ROP) of their cyclized dimer or by the reaction of a polyol, such as a diol, triol, tetraol, or greater with a polycarboxylic acid, such as a diacid, triacid, tetraacid, or greater.

The PEAs can be prepared using methods that include, but are not limited to, ROP of functionalized morpholine-2,5-diones, and polycondensation of functionalized monomers containing reactive groups that include, but are not limited to, hydroxyl, amine, carboxylic, acyl chloride, or ester activated end groups. Galan-Rodriguez, et al., (2011), 3, 65-99.

The molecular weight of the polyester or PEA can vary. In some embodiments, the molecular weight of the polymer is from about 300 Daltons to about 1,000,000 Daltons, preferably 300 Daltons to about 500,000 Daltons, more preferably from about 300 Daltons to about 250,000 Daltons, most preferably from about 300 Daltons to about 100,000 Daltons, most preferably from about 300 Daltons to about 20,000 Daltons. In some embodiments, the minimum molecular weight is 300, 1000, 2,000, 4,000, 5,000, 8,000, or 10,000 Daltons.

The polyester or PEA is substituted with a plurality of radio-opaque graft agents or prepared from an appropriate radio-opaque monomer agent. In some embodiments, the radio-opaque graft agent is covalently bound to the polyester or PEA backbone or covalently bound distal to the polymer backbone via a spacer or linker after preparation of the polymer. In other embodiments, a radio-opaque polyester or PEA is prepared directly via the polymerization of a radio-opaque monomer agent. In some embodiments, the radiopacity of the radio-opaque graft agent and/or the radio-opaque monomer agent is conferred by the incorporation of one or more iodine atoms (e.g., I¹²⁷, I¹²³, and/or I¹³¹) onto the graft agent or monomer.

The degree of substitution of the polyester or PEA with the radio-opaque agent or radio-opaque agent-containing moiety can vary. In some embodiments, the degree of substitution (e.g., the percentage of monomers containing one or more radio-opaque agents or radio-opaque agent containing moieties) is at least about 1%, 2%, 3%, 4%, 5%, 8%, 10%, 15%, 20%, 25%, 30%, 35%, 40%, 45%, 50%, 55%, 60%, 65%, 70%, 75%, 80%, 85%, 90%, or 95%. As the molecular weight of the polymer increases, the iodine content per polymer increases as there are more monomers available for functionalization through grafting or conjugation. The iodine content per polymer also increases as there are more radio-opaque monomers introduced through polymerization. This is contrasted with polymers which are functionalized only at the termini. As the polymer molecular weight increases, the amount of iodine per polymer decreases.

The materials described herein can be used for any application where a radio-opaque material is desired or necessary. In some embodiments, the radio-opaque material, i.e., polyester or PEA including a radio-opaque agent such as iodine, is coated on polymers or metals. In some embodiments, the coated polymers or metals are used to form, whole or in part, a medical device. In some embodiments, the materials are used to form, whole or in part, a medical device. Examples include, but are not limited to, dental implants, breast reconstruction, cranio-maxilofacial implants, soft tissue sutures and staples, abdominal wall repair devices, scaffolds, such as tissue engineering scaffolds, tendon and ligament reconstruction devices, fracture fixation devices, skin, scar, and wrinkle repair/enhancement devices, spinal fixation and fusion devices, nanoparticles, microparticles, and coronary drug eluting stents. The materials can also be used as coatings on medical devices and implants, particularly those used subcutaneously, such as catheters; absorbable constructs for site-specific diagnostic applications; components of absorbable/disintegratable endovascular and urinogenital stents; catheters for deploying radioactive compositions for treating cancer as in the case of iodine-131 (or 123) in the treatment of prostate, lung, intestinal or ovarian cancers; dosage forms for the controlled delivery of iodide in the treatment of thyroid glands and particularly in the case of accidental exposure to radioactive iodine; components of an absorbable device or pharmaceutical product to monitor its pharmacokinetics using iodine-127, 123 or 131; and barrier film to protect surrounding tissues during brachytherapy and similar radiotherapies as in the treatment of ovarian and abdominal cancers.

### BRIEF DESCRIPTION OF THE DRAWINGS

FIG. 1A is a schematic showing preparation of the polyester and PEA polymers with residues that include a radio-opaque agent such as iodine. FIG. 1B shows the preparation of the polyester followed by functionalization of the polymer with a radio-opaque agent-containing moiety. FIG. 1C shows compounds that can be used as initiators of ROP.

FIG. 2 shows the relative X-ray intensities of i-PLA (100%) synthesized using different initiators.

FIG. 3 is a graph showing the x-ray image intensity of polylactide (PLA discs), poly(caprolactone-co-1,4-oxepan-1,5-dione) (PCLOPD) discs, and iodine functionalized P(CLcoOPD) (i-PCL) discs.

FIG. 4 is a graph showing normalized image intensity (%) if non-defected and defected i-PCL.

FIG. 5A is a graph showing the in vitro and in vivo degradation of i-PCL discs on normalized image intensity as a function of time (weeks). FIG. 5B is a graph showing in vitro degradation of i-PCL discs as on molecular weight (kDa) as a function of time (days).

FIG. 6 is a graph showing cell viability (normalized percentage) for PLA and i-PCL films as a function of time (24, 48, and 72 hours).

FIG. 7 shows the relative X-ray intensities of different compositions of mixtures of PLA and iodinated PLA (iPLA) polymers: PLA; 25/75 iPLA,DL-lactide; 50/50 iPLA,DL-lactide; 75/25 iPLA,DL-lactide; 100% iPLA (RXN14).

FIG. 8 shows the nanoparticle size degradation as a function of time (days).

FIG. 9 shows X-ray polymeric pellet degradation (weight percentage) monitored at 12 hours, one day, and three days.

### DETAILED DESCRIPTION OF THE INVENTION

### I. Definitions

“Radio-opaque”, as used herein, refers to materials which stop or reduce passage of x-rays or other radiation through the material. Such materials can be viewed in vivo using X-rays or other radiation.

“Radio-opaque graft agent”, as used herein, refers to a molecule that, when covalently bound to a polymer, renders the resultant material radio-opaque.

“Radio-opaque monomer agent” as used herein, refers to a monomer that results in the formation of a radio-opaque material upon polymerization.

“Molecular weight of the polymer”, as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

“Deep tissue”, as used herein, refers to a tissue depth greater than about 2.5 cm.

“Biodegradable” and “bioresorbable”, are used interchangeably and mean a material that can be decomposed/broken down without requiring removal.

“Biocompatible”, as used herein, refers to materials, or decomposition products thereof, that do not cause an adverse response in vivo.

“Small molecule,” as used herein, refers to molecules with a molecular weight of less than about 2000 g/mol, 1500 g/mol, 1200 g/mol, 1000 g/mol, or 750 g/mol.

“Nanoparticle”, as used herein, generally refers to a particle having a diameter from about 10 nm up to, but not including, about 1 micron, preferably from about 25 nm to about 1 micron. The particles can have any shape and form. Nanoparticles having a spherical shape are generally referred to as “nanospheres”.

“Microparticle”, as used herein, generally refers to a particle having a diameter, such as an average diameter, from about 1 micron to about 100 microns, preferably from about 1 to about 50 microns, more preferably from about 1 to about 30 microns, most preferably from about 1 micron to about 10 microns. The microparticles can have any shape and form. Microparticles having a spherical shape are generally referred to as “microspheres”.

“Mean particle size” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which 90% of the distribution lies within 15% of the median particle size, more preferably within 10% of the median particle size, most preferably within 5% of the median particle size.

“Polydisperse” as used herein, describes a population of nanoparticles or microparticles where 50% of the particle size distribution, more preferably 60% of the particle size distribution, most preferably 75% of the particle size distribution lies within 10% of the median particle size.

“Cross-linking,” or “inter-linking” as generally used herein, means the formation of covalent linkages between a precursor molecule containing one or more nucleophilic groups and a precursor molecule containing one or more electrophilic groups, resulting in an increase in the molecular weight of the material. “Cross-linking” or “inter-linking” may also refer to the formation of covalent bonds via free radical reactions. “Cross-linking” or “inter-linking” may also refer to the formation of non-covalent linkages such as ionic bonds, hydrogen bonds and pi-stacking. The terms “cross-linking” and “inter-linking” are used interchangeably.

### II. Biodegradable, Radio-Opaque Polyesters and Poly(Ester Amides)

Biodegradable, radio-opaque polyesters are described herein. In some embodiments, the polyesters or PEAs are also biocompatible. The polyesters or PEAs contain a plurality of radio-opaque agents or radio-opaque agent-containing moieties that are covalently bound along or from the polymer backbone. The agents/moieties may be bound to the termini of the polymer provided they are bound within the polyester or PEA backbone as well. The polyester or PEA can be aliphatic, aromatic, or combinations thereof. The aliphatic and/or aromatic polyester or PEA can also include saturated and/or unsaturated groups on the backbone and/or side chains. The polyester or PEA can contain one (e.g., homopolymer), two (e.g., copolymer), three (e.g., terpolymer) or more different monomer units. In addition, the monomers in the polyester or PEA can be arranged randomly, in blocks or in alternating order. The polyester or PEA can be amphiphilic, hydrophilic or hydrophobic. The polyester or PEA can be positively charged, negatively charged or neutral.

The polyesters can be prepared using methods known in the art including, but not limited to, the reaction of one or more hydroxy acids (e.g., glycolide, lactide, caprolactone) or by ROP of their cyclized dimer or by the reaction of a polyol, such as a diol, triol, tetraol, or greater with a polycarboxylic acid, such as a diacid, triacid, tetraacid, or greater.

The PEA can be prepared using methods that include, but are not limited to, ROP of functionalized morpholine-2,5-diones, and polycondensation of functionalized monomers with reactive groups that include, but are not limited to, hydroxyl, amine, carboxylic, acyl chloride, or ester activated end groups. Galan-Rodriguez, et al., (2011), 3, 65-99.

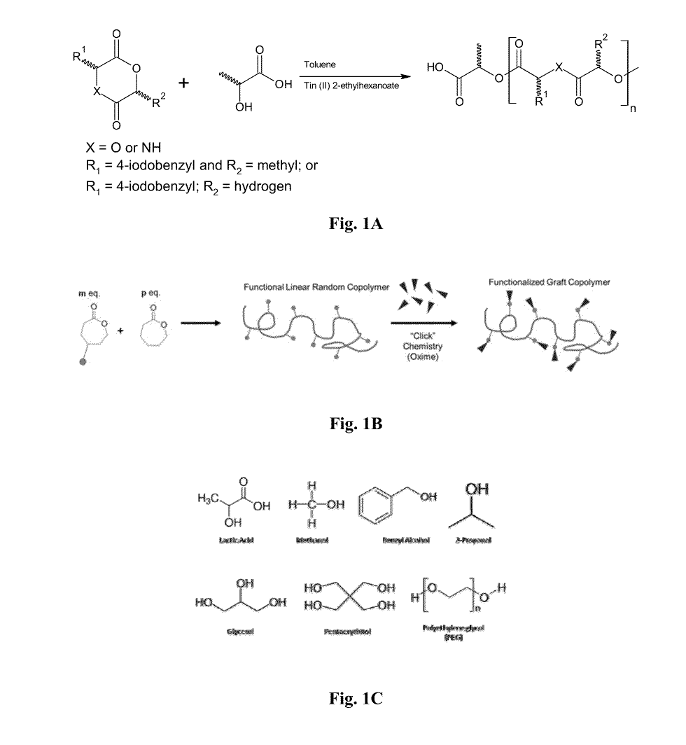

A general approach to prepare the polyesters and PEAs via ROP is shown in FIG. 1. The radio-opaque agent can be incorporated into a monomer prior to ring-opening, or the radio-opaque agent can be incorporated after the polymer has been formed. FIG. 1A shows an embodiment in which the radio-opaque agent is incorporated into the monomer prior to ROP. A polyester is generated when X is oxygen, while a PEA is generated when X is —NH— from the six-membered ring. In a preferred embodiment, R₁ and R₂ are 4-iodobenzyl and methyl, respectively. In another preferred embodiment, R₁ and R₂ are 4-iodobenzyl and hydrogen, respectively. In these preferred embodiments, the initiator of ROP is D/L-lactide, the solvent is toluene and the catalyst tin(II) 2-ethylhexanoate (tin(II) octanoate). FIG. 1B shows an embodiment in which the radio-opaque agent is incorporated after the polymer has been generated. In a preferred embodiment, ε-caprolactone containing a functionalizable group is polymerized in the presence of another ε-caprolactone that does not include a functionalizable group. In a preferred embodiment, the radio-opaque agent is attached to the polymer via a method that includes, but is not limited to, oxime “click” chemistry. FIG. 1C shows different initiators of ROP. These non-limiting examples show initiators with one nucleophile (lactic acid, methanol, benzyl alcohol and 2-propanol), two nucleophiles (PEG), three nucleophiles (glycerol) and four nucleophiles (pentaerythitol).

A. Polyesters

Exemplary polyesters include, but are not limited to, those formed from hydroxy acids including but not limited to, lactide, glycolide, caprolactone, trimethylene carbonate, p-dioxanone,1,5-dioxepan-2-one, morpholinedione, polyhydroxyalkanoate, such as polyhydroxybutyrate (P3HB), poly(4-hydroxybutyrate) (P4HB), poly(3-hydroxyvalerate) and copolymers thereof, polyesters formed from an aliphatic or aromatic diacid and an aliphatic or aromatic diol, including but not limited to, polyethylene adipate, polybutylene succinate, polyethylene terephthalate, polybutylene terephthalate, polytrimethylene terephthalate, polyethylene naphthalate, aliphatic or aromatic polyesters formed from two hydroxy carboxylic acid, including but not limited to, Vectran (formed from 4-hydroxybenzoic acid and 6-hydroxynaphthalene-2-carboxylic acid) and combinations thereof. The polyesters can be generated with variable hydrophilicities, aliphatic/aromatic ratio, and saturation/unsaturation ratios.

Amphiphilic polyesters can be formed from block co-polymers of esters, or from the incorporation of biocompatible hydrophilic or hydrophobic polymers into hydrophobic or hydrophilic polyesters, respectively.

The molecular weight of the polyester can vary. In some embodiments, the molecular weight of the polymer is from about 300 Daltons to about 1,000,000 Daltons, preferably 300 Daltons to about 500,000 Daltons, more preferably from about 300 Daltons to about 250,000 Daltons, most preferably from about 300 Daltons to about 100,000 Daltons, most preferably from about 300 Daltons to about 20,000 Daltons. In some embodiments, the minimum molecular weight is 2,000, 4,000, 5,000, 8,000, or 10,000 Daltons.

B. Poly(Ester Amides)

PEA are polymers that have both ester and amide functional groups on their backbone. The PEA can be aliphatic, aromatic, or combinations thereof. The aliphatic and/or aromatic PEA can also include saturated and/or unsaturated groups on the backbone and/or side chains. The PEAs can be generated with variable hydrophilicities, ester/amide ratios, aliphatic/aromatic ratio, and saturation/unsaturation ratios. They can be formed from synthetic routes that include, but are not limited to, ROP of morpholine-2,5-diones, and polycondensation of monomers that include reactive, hydroxyl, amine, carboxylic, acyl chloride, or ester activated end groups, or combinations thereof.

The morpholine-2,5-diones can be obtained from the cyclization of N-(α-haloacyl)-α-amino acid, intramolecular transesterification N-(α-hydroxyacyl)-α-amino acid esters, and O-(α-aminoacyl)-α-hydroxy acid esters. Galan-Rodriguez, et al., (2011), 3, 65-99.

The α-haloacyl and α-hydroxyacyl monomers can be obtained from acyl halides shown by Formula I below:

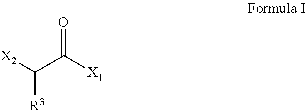

wherein X₁ is a hydroxyl group, —OR⁴, or halogen, wherein the halogen is preferably chlorine. R⁴ is alkyl, alkenyl, alkynyl, aryl, alkylaryl, cycloalkyl, heterocycloalkyl, heteroaryl group.

X₂ is a hydroxyl group or halogen, wherein the halogen is preferably chlorine or bromine.

R³ is hydrogen, or alkyl, alkenyl, alkynyl, aryl, alkylaryl, cycloalkyl, heterocycloalkyl, heteroaryl group substituted or unsubstituted with sulfhydryl, hydroxy, amino, cyano, nitro, azide, aldehyde, ester, sulfonate ester, isocyanate, thioisocyanate and carboxylic acid.

The amino acids that can be used to prepare the morpholine-2,5-diones include natural amino acids, unnatural amino acids, modified amino acids, protected amino acids or mimetics of amino acids. In some embodiments, the amino acids include lysine, ornithine, serine, cysteine, selenocysteine, arginine, aspartic acid, glutamic acid, phenylalanine, tyrosine, 3,4-dihydroxyphenylalanine, tryptophan, 2-allylgycine, and threonine. These amino acids can be the L- or D-stereoisomers, and can be functionalized to include a radio-opaque agent.

Polycondensation can be used to prepare PEAs through the reaction of (i) diamide-diols, diester-diamides, and ester-diamine monomers with dicarboxylic acids or activated derivatives of dicarboxylic acids; (ii) diamide-diester monomers with diols or aminoalcohols; and (iii) acid anhydrides, dicarboxylic acid derivatives with aminoalcohols. The PEAs can be functionalized by incorporating amino acids including, but not limited to, α-amino acids and ω-amino acids. Galan-Rodriguez, et al., (2011), 3, 65-99. These amino acids can be the L- or D-stereoisomers, and can be functionalized to include a radio-opaque agent.

The molecular weight of the PEA can vary. In some embodiments, the molecular weight of the polymer is from about 300 Daltons to about 1,000,000 Daltons, preferably 300 Daltons to about 500,000 Daltons, more preferably from about 300 Daltons to about 250,000 Daltons, most preferably from about 300 Daltons to about 100,000 Daltons, most preferably from about 300 Daltons to about 20,000 Daltons. In some embodiments, the minimum molecular weight is 300, 1000, 2,000, 4,000, 5,000, 8,000, or 10,000 Daltons.

Amphiphilic poly(ester amides) can be formed from block co-polymers of ester amides, or from the incorporation of biocompatible hydrophilic or hydrophobic polymers into hydrophobic or hydrophilic poly(ester amides), respectively.

C. Hydrophilic Polymers

Suitable hydrophilic polymers include, but are not limited to, hydrophilic polypeptides, such as poly-L-glutamic acid, gamma-polyglutamic acid, poly-L-aspartic acid, poly-L-serine, or poly-L-lysine, poly(alkylene glycols) such as polyethylene glycol (PEG), poly(propylene glycol) and copolymers of ethylene glycol and propylene glycol, poly(oxyethylated polyol), poly(olefinic alcohol), polyvinylpyrrolidone), poly(hydroxyalkylmethacrylamide), poly(hydroxyalkylmethacrylate), poly(saccharides), poly(hydroxy acids), poly(vinyl alcohol), as well as copolymers thereof. In some embodiments, the hydrophilic polymer is PEG.

D. Hydrophobic Polymers

Suitable hydrophobic polymers include, but are not limited to, polyhydroxyacids, polyhydroxyalkanoates, polycaprolactones, poly(orthoesters); polyanhydrides, poly(phosphazenes), polycarbonates, polyamides, polyesteramides, polyesters, poly(alkylene alkylates), hydrophobic polyethers, polyurethanes, polyetheresters, polyacetals, polycyanoacrylates, polyacrylates, polymethylmethacrylates, polysiloxanes, polyketals, polyhydroxyvalerates, polyalkylene oxalates, polyalkylene succinates, and copolymers thereof.

E. Radio-Opaque Agents

The polyester is substituted with a plurality of radio-opaque graft agents or prepared from an appropriate radio-opaque monomer agent. In some embodiments, the radio-opaque graft agent is covalently bound to the polyester or PEA backbone or covalently bound distal to the polymer backbone via a spacer or linker after preparation of the polymer. In other embodiments, a radio-opaque polyester or PEA is prepared directly via the polymerization of a radio-opaque monomer agent. In some embodiments, the radiopacity of the radio-opaque graft agent and/or the radio-opaque monomer agent is conferred by the incorporation of one or more iodine atoms (e.g., I¹²⁷, I¹²³, and/or I¹³¹) onto the graft agent or monomer.

In some embodiments, the radio-opaque graft agent is an iodinated hydroxylamine. In particular embodiments, the hydroxylamine contains an aromatic moiety, such as a benzene ring. In more particular embodiments, the radio-opaque graft agent is O-(2-iodobenzyl)hydroxylamine. In other embodiments, the radio-opaque grafting agent is, but is not limited to: O-(2,3,5-triiodobenzyl)hydroxylamine, O-(2-iodohomobenzyl)hydroxylamine, O-(2,3,5-triiodo-homobenzyl)hydroxylamine, (2-iodophenyl)methanethiol, (2,3,5-triiodophenyl)methanethiol, (2-iodophenyl)ethanethiol, (2,3,5-triiodophenyl)ethanethiol, 2-iodo-benzylhydrazine, 2,3,5-triiodobenzylhydrazine, m-iodo-homobenzylhydrazine, 2,3,5-triiodohomobenzylhydrazine, 2-iodo-benzylamine, 2,3,5-triiodobenzylamine, m-iodohomobenzylamine, 2,3,5-triiodo-homobenzylamine, 4-(2-iodobenzyl)semicarbazide, 4-(2,3,5-triiodobenzyl)semicarbazide, 4-(m-iodohomobenzyl)semicarbazide, 4-(2,3,5-triiodohomobenzyl)semicarbazide, 2-(2-iodobenzyl)semicarbazide, 2-(2,3,5-triiodobenzyl)semicarbazide, 2-(m-iodohomobenzyl)semicarbazide, and 2-(2,3,5-triiodohomobenzyl)semicarbazide.

In more general embodiments, the radio-opaque grafting agent is a mono- or multi-iodinated (in any substitution pattern) mono- or multi-cyclic aromatic or heteroaromatic moiety connected by an intervening linker of any length and composition to a suitable nucleophile to facilitate grafting. Examples of mono- or multi-iodinated aromatic moieties include, but are not limited to, substituted or unsubstituted benzene, naphthalene, anthracene, phenanthrene; furan, thiophene, pyrrole, benzofuran, benzothiophene, indole, pyridine, quinoline, isoquinoline, phenanthroline, imidazole, benzimidazole, purine, pyrimidine, pyridazine, pyrazine, 1,2,4-triazine, 1,2,3-triazine, pyrazole, 1,2,4-triazole, 1,2,4-triazole, isoxazole, oxazole, thiazole, and isothiazole; and the nucleophile is, but is not limited to: hydroxylamine, hydrazine, alcohol, thiol, amine, and semicarbazide.

In another general embodiment, the radio-opaque grafting agent is a mono- or multi-iodinated (in any substitution pattern) mono- or multi-cyclic aromatic or heteroaromatic moiety connected by an intervening aliphatic linker of any length to a suitable electrophile to facilitate grafting, wherein the aromatic and heteroaromatic moieties are defined as in the previous embodiment, and the electrophile is, but is not limited to: an alkyl halide, alkyl sulfonate, acyl halide, carboxylic acid, or ester. Further embodiments may incorporate the following iodinated molecules in a suitable radio-opaque grafting agent: (Diacetoxyiodo)benzene, [Hydroxy(tosyloxy)iodo]benzene, Bis(2,4,6-trimethylpyridine)iodine(I) hexafluorophosphate, Bis(tertbutylcarbonyloxy)iodobenzene, L-Thyroxine, 2,3,5-Triiodobenzoic acid, and [Bis(trifluoroacetoxy)iodo]pentafluorobenzene.

In some embodiments, the radio-opaque monomer agent is an iodine-containing lactide. In specific embodiments, the radio-opaque monomer agent is 4-iodobenzyl lactide. In other embodiments, the radio-opaque monomer agent is 4-iodobenzyl glycolide, 3-(4-iodobenzyl) caprolactone, 4-iodophenylalanine. In a general embodiment, the radio-opaque monomer agent is, but is not limited to, a mono- or multi-iodinated (in any substitution pattern) mono- or multi-cyclic aromatic or heteroaromatic moiety connected by an intervening linker of any length and composition to the core lactide, glycolide, ε-caprolactone, or amino acid scaffold. In the previous general embodiment, the mono- or multi-iodinated aromatic moiety is, but is not limited to, substituted or unsubstituted: benzene, naphthalene, anthracene, and phenanthrene; and the mono- or multi-iodinated heteroaromatic is, but is not limited to, unsubstituted or substituted forms of: furan, thiophene, pyrrole, benzofuran, benzothiophene, indole, pyridine, quinoline, isoquinoline, phenanthroline, imidazole, benzimidazole, purine, pyrimidine, pyridazine, pyrazine, 1,2,4-triazine, 1,2,3-triazine, pyrazole, 1,2,4-triazole, 1,2,4-triazole, isoxazole, oxazole, thiazole, and isothiazole. In some embodiments, the radio-opaque graft agent or radio-opaque monomer agent is a synthetic molecule or its derivative, a natural molecule, or a combination.

In some embodiments, the radio-opaque agent is incorporated directly into the polymer backbone containing mono- or multi-iodinated aromatic or heteroaromatic monomers on the backbone. Exemplary iodinated aromatic monomers are those represented by the Formula II below:

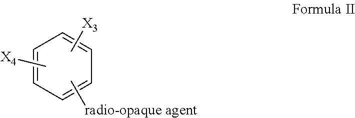

wherein X₃ and X₄ are independently amine, C₁-C₁₀ amine, amide, C₁-C₁₀ amide, carboxylic acid, C₁-C₁₀ carboxylic acid, ester, C₁-C₁₀ ester, aldehyde, C₁-C₁₀ aldehyde, C₁-C₁₀ thiol, hydroxyl, C₁-C₁₀ hydroxyl, C₁-C₁₀ alkene, alkyne, nitro, C₁-C₁₀ nitro, isocyanate, C₁-C₁₀ isocyanate, thioisocyanate, C₁-C₁₀ thioisocyanate, cyano, and C₁-C₁₀ cyano. In some embodiments, the radio-opaque agent is iodine. Examples include, but are not limited to, 3-iodo-1,5-dibenzoic acid, 2-iodo-4-nitrobenzoic acid, 3-iodo-4-nitrobenzoic acid, 2-iodo-4-aminobenzoic acid, 3-iodo-4-cyanobenzoic acid, 3-hydroxy-5-iodobenzoic acid, and methyl 3-amino-5-iodobenzoate, 3-amino-5-iodophenylacetic acid, methyl 2-(aminomethyl)-5-iodobenzoate, 3-formyl-4-iodobenzoic acid, 5-cyano-2-iodobenzoic acid, ethyl 3-amino-5-iodophenylacetate, 3-amino-5-iodobenzamide, 5-nitro-3-iodobenzamide. In some embodiments, the aromatic group is monoaryl, polyaryl, heteroaromatic, or combinations thereof.

In some embodiments, the iodine-containing moiety is an iodine-containing hydroxylamine. In particular embodiments, the hydroxylamine contains an aromatic moiety, such as a benzene ring. In more particular embodiments, the iodine-containing moiety is O-(2-iodobenzyl)hydroxylamine.

The degree of substitution of the polyester or PEA with the radio-opaque agent or radio-opaque agent-containing moiety can vary. In some embodiments, the degree of substitution (e.g., the percentage of monomers containing one or more radio-opaque agents or radio-opaque agent containing moieties) is at least about 1%, 2%, 3%, 4%, 5%, 8%, 10%, 15%, 20%, 25%, 30%, 35%, 40%, 45%, 50%, 55%, 60%, 65%, 70%, 75%, 80%, 85%, 90%, 95%, or 99%. In some embodiments, the degree of substitution is 100%. As the molecular weight of the polymer increases, the iodine content per polymer increases as there are more monomers available for functionalization through grafting or conjugation. The iodine content per polymer also increases as there are more radio-opaque monomers introduced through polymerization. This is contrasted with polymers which are functionalized only at the termini. As the polymer molecular weight increases, the amount of iodine per polymer decreases.

F. Additional Imaging Agents

Other imaging agents can also be incorporated in, or attached to, the polymers described herein in the manner discussed below. These agents can be in addition to or in place of radio-opaque agents, such as iodine. Examples include, but are not limited to, Gadolinium (contrast agent that may be given during MRI scans; highlights areas of tumor or inflammation); PET and Nuclear Medicine Imaging Agents, such as 64Cu-ATSM (64Cu diacetyl-bis(N4-methylthiosemicarbazone), FDG (18F-fluorodeoxyglucose, radioactive sugar molecule, that, when used with PET imaging, produces images that show the metabolic activity of tissues); 18F-fluoride (imaging agent for PET imaging of new bone formation); FLT (3′-deoxy-3′-[18F]fluorothymidine, radiolabeled imaging agent that is being investigated in PET imaging for its ability to detect growth in a primary tumor); FMISO (18F-fluoromisonidazole, imaging agent used with PET imaging that can identify hypoxia (low oxygen) in tissues); Gallium (attaches to areas of inflammation, such as infection and also attaches to areas of rapid cell division, such as cancer cells); Technetium-99m (radiolabel many different common radiopharmaceuticals; used most often in bone and heart scans); Thallium (radioactive tracer typically used to examine heart blood flow); and combinations thereof. The concentration of the agents can be the same as described above.

G. Cross-Linking or Inter-Linking of Polymers

The presence of unsaturated groups and/or reactive functional groups on the back bone and/or side chains of the polymers described herein provide the ability to cross-link the polymers to form networks of polymers.

In some embodiments, the polyesters are cross-linked or inter-linked with another polyester that contains or lacks a radio opaque agent. In some embodiments, the polyesters are cross-linked or inter-linked with hydrophilic, hydrophobic or amphiphilic polymers. In some embodiments, the polyesters are cross-linked or inter-linked with small molecules. In some embodiments, the polyesters are mixed with another hydrophilic, hydrophobic or amphiphilic polymer.

In some embodiments, the PEAs are cross-linked or inter-linked with another PEA that contains or lacks a radio opaque agent. In some embodiments, the PEAs are cross-linked or inter-linked with hydrophilic, hydrophobic or amphiphilic polymers. In some embodiments, the PEAs are cross-linked or inter-linked with small molecules. In some embodiments, the PEAs are mixed with another hydrophilic, hydrophobic or amphiphilic polymer.

A common strategy to cross-link or inter-link polymers is via the use of chemical cross-linking or inter-linking agents. In some embodiments, the cross-linking or inter-linking agents are small molecules, monomers, dimers, polymers, or combinations thereof. In some embodiments, the cross-linkers are homo-bifunctional, hetero-bifunctional, homo-polyfunctional or hetero-polyfunctional.

In some embodiments, the cross-linkers have the structures shown below in Formula III:

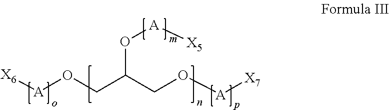

### or Formula IV:

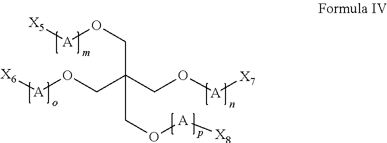

wherein A is —(CH₂)₂O— or hydrogen,
wherein m, n, o and p are independently integers from 1-50, and
wherein, as valence permits, X₅, X₆, X₇, and X₈, when present, are independently

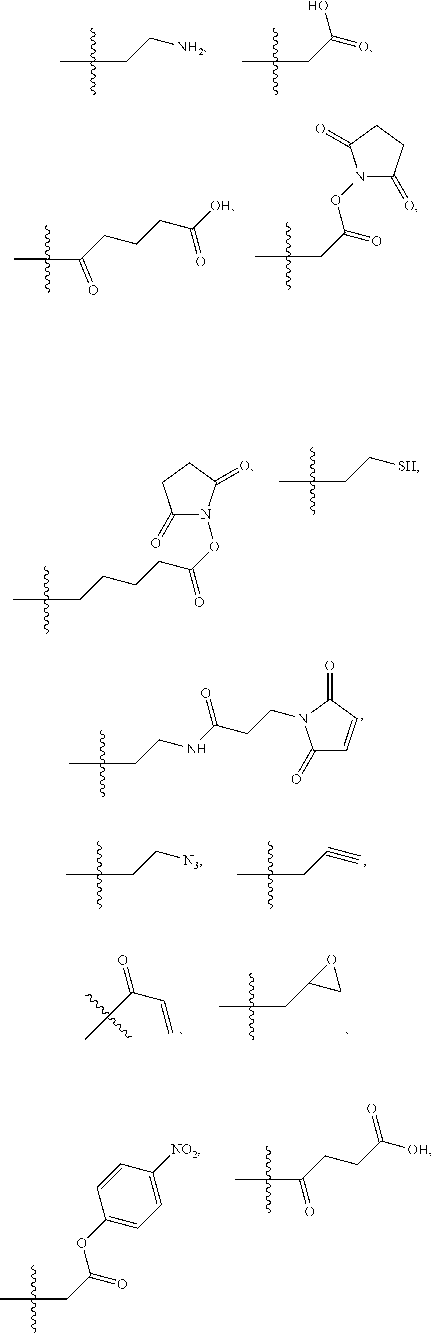

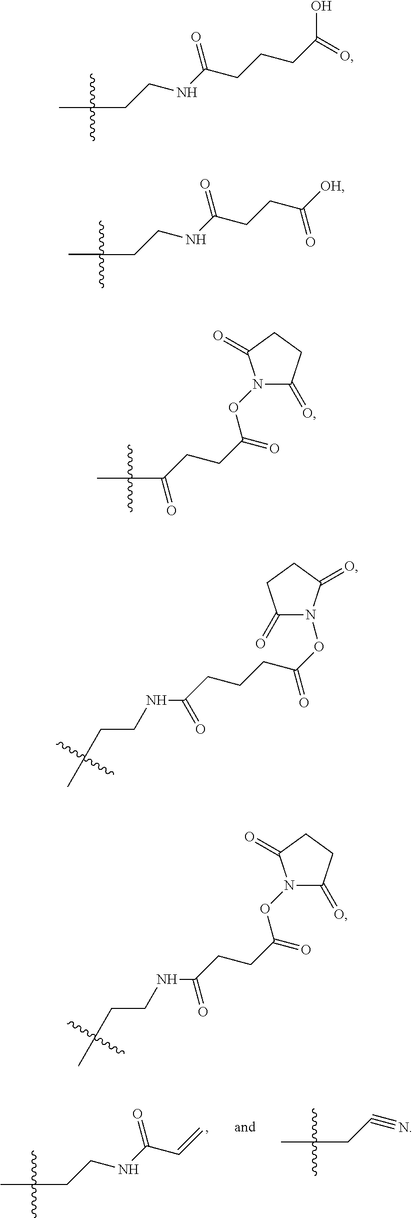

In some embodiments, X₅, X₆, X₇, and X₈, when present, are the same giving rise to homo-polyfunctional cross-linkers. Additional examples of homo-polyfunctional cross-linkers include, but are not limited to, glycerol, monosaccharides, disaccharides, polysaccharides, hyperbranched polyglycerol, polyethylenimine, poly(amido amine), trimethylol propane, trimethylol propane triacrylate, triethanolamine, glycerol trisglutaroyl chloride, poly(amino acids) such as poly-L-lysine, poly-L-ornithine, poly-L-aspartic acid, poly-L-glutamic acid and poly-L-serine. EP 2,322,227 by Universidade de Santiago de Compostela describes dendrimers containing azide groups, the contents of which are incorporated herein by reference. The azides can be reduced to amines that are also crosslinkers.

In some embodiments, X₅, X₆, X₇, and X₈, when present, are different giving rise to hetero-polyfunctional cross-linkers. Additional examples of hetero-polyfunctional cross-linkers include, but are not limited to, 2-aminomalonaldehyde, genipin, 2,3-dithiopropanol, 2,3-bis(thiomethyl)butan-1,4-diol, 2,3-dihydroxybutane-1,4-dithiol, methyl 3,4,5-trihydroxybenzoate, tris(hydroxymethyl)aminomethane and citric acid.

Examples of homo-bifunctional cross-linking agents include, but are not limited to, aldehydes such as ethanedial, pyruvaldehyde, 2-formylmalonaldehyde, glutaraldehyde, adipaldehyde, heptanedial, octanedial; di-glycidyl ether, diols such as 1,2-ethanediol, 1,3-propanediol, 1,4-butanediol, 2,3-butanediol, 1,5-pentanediol, benzene-1,4-diol, 1,6-hexanediol, tetra(ethylene glycol)diol), PEG, di-thiols such as 1,2-ethanedithiol, 1,3-propanedithiol, 1,4-butanedithiol, 2,3-butanedithiol, 1,5-pentanedithiol, benzene-1,4-dithiol, 1,6-hexanedithiol, tetra(ethylene glycol)dithiol), di-amine such as ethylene diamine, propane-1,2-diamine, propane-1,3-diamine, N-methylethylenediamine, N,N′-dimethylethylenediamine, pentane-1,5-diamine, hexane-1,6-diamine, spermine and spermidine, divinyladipate, divinylsebacate, diamine-terminated PEG, double-ester PEG-N-hydroxysuccinimide, and di-isocyanate-terminated PEG.

Examples of hetero-bifunctional linkers include, but are not limited to, epichlorohydrin, S-acetylthioglycolic acid N-hydroxysuccinimide ester, 5-azido-2-nitrobenzoic acid N-hydroxysuccinimide ester, 4-azidophenacyl bromide, bromoacetic acid N-hydroxysuccinimide ester, N-(3-dimethylaminopropyl)-N′-ethylcarbodiimide, Iodoacetic acid N-hydroxysuccinimide ester, 4-(N-maleimido)benzophenone 3-(2-pyridyldithio)propionic acid N-hydroxysuccinimide ester 3-maleimidobenzoic acid N-hydroxysuccinimide ester, N,N′-cystamine-bis-acrylamide, N,N′-methylene-bis-acrylamide and N,N′-ethylene-bis-acrylamide.

In some embodiments, cross-linkers are also the reaction product of reactants including, but not limited to, a diisocyanate, a diamine and a polyetherdiol. Examples of reactants include but not limited to aliphatic diisocyanate selected from the group consisting of 1,4-tetramethylene diisocyanate, 1,4-bis(meth ylene isocyanato)cyclohexane, 1,6-hexamethylene diisocyanate, and lysine diisocyanate.

Cross-linking can also be accomplished using enzymatic means; for example, transglutaminase has been approved as a GRAS substance for cross-linking seafood products. Cross-linking can be initiated by physical means such as thermal treatment, UV irradiation and gamma irradiation.

H. Initiators of Ring Opening Polymerization

ROP can be carried out using any method known in the art, such as anionic ROP (AROP). AROP involves a nucleophilic attack of a charged or uncharged nucleophile on the carbonyl carbon or on the carbon atom next to an acyl-oxygen, resulting in the opening of the ring, and the formation of another charged or uncharged nucleophile. The charged or uncharged nucleophile attacks a carbonyl carbon or a carbon atom next to the acyl oxygen of another cyclic monomer, resulting in the propagation of the polymer.

The structure of the initiator can determine the architecture of the growing polymer: initiators with one nucleophile, i.e., monovalent, give rise to linear polymers; initiators with two or more nucleophiles, i.e., divalent or multivalent, respectively, give rise to branched, star-shaped, brush-shaped, comb-shaped, ladder-shaped, hyperbranched, dendrimeric polymers, or combinations thereof. Any of the cross-linking agents described herein, which contain nucleophiles can be used as initiators of ROP. In some embodiments, the cross-linking agents contain functional groups that can be reduced to generate nucleophiles. For example carboxylic acids, aldehydes, esters, acyl halides can be reduced alcohols; cyano groups and azides can be reduced to amines; and disulfides can be reduced to thiols. Additional examples of initiators include, but are not limited to, glycerol, monosaccharides, disaccharides, polysaccharides, hyperbranched polyglycerol, polyethylenimine, poly(amido amine), trimethylol propane, triethanolamine, poly(amino acids) such as poly-L-lysine, poly-L-ornithine, poly-L-aspartic acid, poly-L-glutamic acid and poly-L-serine, genipin, 2,3-dithiopropanol, 2,3-bis(thiomethyl)butan-1,4-diol, 2,3-dihydroxybutane-1,4-dithiol, methyl 3,4,5-trihydroxybenzoate, tris(hydroxymethyl)aminomethane, citric acid, 1,2-ethanediol, 1,3-propanediol, 1,4-butanediol, 2,3-butanediol, 1,5-pentanediol, benzene-1,4-diol, 1,6-hexanediol, tetra(ethylene glycol)diol), PEG, di-thiols such as 1,2-ethanedithiol, 1,3-propanedithiol, 1,4-butanedithiol, 2,3-butanedithiol, 1,5-pentanedithiol, benzene-1,4-dithiol, 1,6-hexanedithiol, tetra(ethylene glycol)dithiol), di-amine such as ethylene diamine, propane-1,2-diamine, propane-1,3-diamine, N-methylethylenediamine, N,N′-dimethylethylenediamine, pentane-1,5-diamine, hexane-1,6-diamine, spermine and spermidine.

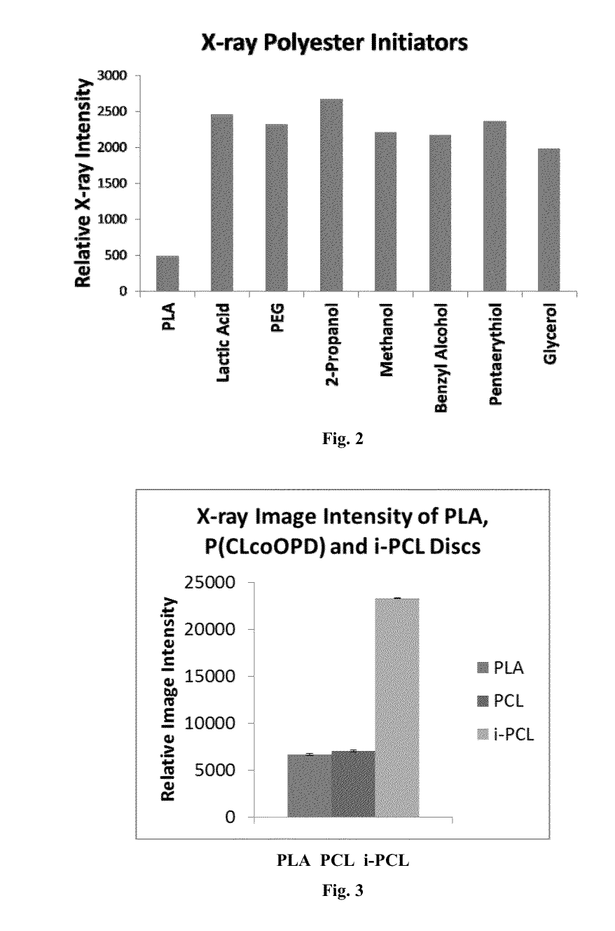

Several initiators were used to carry out the ROP reactions described herein. In some embodiments, the initiator is PLA, lactic acid, PEG, 2-propanol, methanol, benzyl alcohol, pentaerythitol and glycerol. Surprisingly, the initiator used can have an effect on the X-ray image intensity of the polymers. Referring to FIG. 2, polymers form from ROP initiated with PLA showed in lowest relative X-ray intensity compared to the other initiators. In a preferred embodiment, the initiator is lactic acid, PEG, 2-propanol, methanol, benzyl alcohol, pentaerythitol or glycerol.

Also contemplated are initiators of ROP that proceed via cationic ROP, metal coordination-insertion ROP and radical ROP mechanisms.

I. Microparticles and Nanoparticles

The polyesters or PEAs described herein can be used to prepare microparticles and/or nanoparticles of any shape, size and form. In some embodiments, the polyesters or PEAs described herein are used to form the particles, i.e., the polyesters or PEAs form the shell of the particle. In other embodiments, the polyesters or PEAs described herein are encapsulated within one or more biocompatible polymers to form the particles. The particles can further contain additional components, such as one or more therapeutic, prophylactic, and/or diagnostic agents.

Exemplary biocompatible polymers include, but are not limited to, Examples of biocompatible polymers include but are not limited to polystyrenes; poly(hydroxy acid); poly(lactic acid); poly(glycolic acid); poly(lactic acid-co-glycolic acid); poly(lactic-co-glycolic acid); poly(lactide); poly(glycolide); poly(lactide-co-glycolide); polyanhydrides; polyorthoesters; polyamides; polycarbonates; polyalkylenes; polyethylenes; polypropylene; polyalkylene glycols; poly(ethylene glycol); polyalkylene oxides; poly(ethylene oxides); polyalkylene terephthalates; poly(ethylene terephthalate); polyvinyl alcohols; polyvinyl ethers; polyvinyl esters; polyvinyl halides; polyvinyl chloride); polyvinylpyrrolidone; polysiloxanes; polyvinyl alcohols); poly(vinyl acetate); polyurethanes; co-polymers of polyurethanes; derivatized celluloses; alkyl cellulose; hydroxyalkyl celluloses; cellulose ethers; cellulose esters; nitro celluloses; methyl cellulose; ethyl cellulose; hydroxypropyl cellulose; hydroxypropyl methyl cellulose; hydroxybutyl methyl cellulose; cellulose acetate; cellulose propionate; cellulose acetate butyrate; cellulose acetate phthalate; carboxylethyl cellulose; cellulose triacetate; cellulose sulfate sodium salt; polymers of acrylic acid; methacrylic acid; copolymers of methacrylic acid; derivatives of methacrylic acid; poly(methyl methacrylate); poly(ethyl methacrylate); poly(butylmethacrylate); poly(isobutyl methacrylate); poly(hexylmethacrylate); poly(isodecyl methacrylate); poly(lauryl methacrylate); poly(phenyl methacrylate); poly(methyl acrylate); poly(isopropyl acrylate); poly(isobutyl acrylate); poly(octadecyl acrylate); poly(butyric acid); poly(valeric acid); poly(lactide-co-caprolactone); copolymers of poly(lactide-co-caprolactone); blends of poly(lactide-co-caprolactone); hydroxyethyl methacrylate (HEMA); copolymers of HEMA with acrylate; copolymers of HEMA with polymethylmethacrylate (PMMA); polyvinylpyrrolidone/vinyl acetate copolymer (PVP/VA); acrylate polymers/copolymers; acrylate/carboxyl polymers; acrylate hydroxyl and/or carboxyl copolymers; polycarbonate-urethane polymers; silicone-urethane polymers; epoxy polymers; cellulose nitrates; polytetramethylene ether glycol urethane; polymethylmethacrylate-2-hydroxyethylmethacrylate copolymer; polyethylmethacrylate-2-hydroxyethylmethacrylate copolymer; polypropylmethacrylate-2-hydroxyethylmethacrylate copolymer; polybutylmethacrylate-2-hydroxyethylmethacrylate copolymer; polymethylacrylate-2-hydroxyethylmethacrylate copolymer; polyethylacrylate-2-hydroxyethylmethacrylate copolymer; polypropylacrylate-2-hydroxymethacrylate copolymer; polybutylacrylate-2-hydroxyethylmethacrylate copolymer; copolymermethylvinylether maleic anhydride copolymer; poly(2-hydroxyethyl methacrylate) acrylate polymer/copolymer; acrylate carboxyl and/or hydroxyl copolymer; olefin acrylic acid copolymer; ethylene acrylic acid copolymer; polyamide polymers/copolymers; polyimide polymers/copolymers; ethylene vinylacetate copolymer; polycarbonate urethane; silicone urethane; polyvinylpyridine copolymers; polyether sulfones; polygalactia poly-(isobutyl cyanoacrylate), and poly(2-hydroxyethyl-L-glutamine); polydimethyl siloxane; poly(caprolactones); poly(ortho esters); polyamines; polyethers; polyesters; poly(ester amides); polycarbamates; polyureas; polyimides; polysulfones; polyacetylenes; polyethyeneimines; polyisocyanates; polyacrylates; polymethacrylates; polyacrylonitriles; polyarylates; and combinations, copolymers and/or mixtures of two or more of any of the foregoing.

The biodegradable polymer can contain a synthetic polymer, although natural polymers also can be used. The polymer can be, for example, poly(lactic-co-glycolic acid) (PLGA), polystyrene or combinations thereof. The polystyrene can, for example, be modified with carboxyl groups. Other examples of biodegradable polymers include poly(hydroxy acid); poly(lactic acid); poly(glycolic acid); poly(lactic acid-co-glycolic acid); poly(lactide); poly(glycolide); poly(lactide-co-glycolide); polyanhydrides; polyorthoesters; polyamides; polycarbonates; polyalkylenes; polyethylene; polypropylene; polyalkylene glycols; poly(ethylene glycol); polyalkylene oxides; poly(ethylene oxides); polyalkylene terephthalates; poly(ethylene terephthalate); polyvinyl alcohols; polyvinyl ethers; polyvinyl esters; polyvinyl halides; polyvinyl chloride); polyvinylpyrrolidone; polysiloxanes; poly(vinyl alcohols); polyvinyl acetate); polyurethanes; co-polymers of polyurethanes; derivatized celluloses; alkyl cellulose; hydroxyalkyl celluloses; cellulose ethers; cellulose esters; nitro celluloses; methyl cellulose; ethyl cellulose; hydroxypropyl cellulose; hydroxypropyl methyl cellulose; hydroxybutyl methyl cellulose; cellulose acetate; cellulose propionate; cellulose acetate butyrate; cellulose acetate phthalate; carboxylethyl cellulose; cellulose triacetate; cellulose sulfate sodium salt; polymers of acrylic acid; methacrylic acid; copolymers of methacrylic acid; derivatives of methacrylic acid; poly(methyl methacrylate); poly(ethyl methacrylate); poly(butylmethacrylate); poly(isobutyl methacrylate); poly(hexylmethacrylate); poly(isodecyl methacrylate); poly(lauryl methacrylate); poly(phenyl methacrylate); poly(methyl acrylate); poly(isopropyl acrylate); poly(isobutyl acrylate); poly(octadecyl acrylate); poly(butyric acid); poly(valeric acid); poly(lactide-co-caprolactone); copolymers of poly(lactide-co-caprolactone); blends of poly(lactide-co-caprolactone); polygalactin; poly-(isobutyl cyanoacrylate); poly(2-hydroxyethyl-L-glutamine); and combinations, copolymers and/or mixtures of one or more of any of the foregoing.

As used herein, “derivatives” include polymers having substitutions, additions of chemical groups and other modifications routinely made by those skilled in the art. For example, functional groups on the polymer can be capped to alter the properties of the polymer and/or modify (e.g., decrease or increase) the reactivity of the functional group. For example, the carboxyl termini of carboxylic acid containing polymers, such as lactide- and glycolide-containing polymers, may optionally be capped, e.g., by esterification, and the hydroxyl termini may optionally be capped, e.g. by etherification or esterification.

J. Therapeutic, Prophylactic, and/or Diagnostic Agents

The polymers described herein can be formulated with one or more therapeutic, prophylactic, and/or diagnostic agents. The agents can be mixed with the polyesters or PEAs, incorporated into microparticles and/or nanoparticles formed of the polyesters or PEAs and/or containing the polyesters or PEAs, or covalently or ionically associated with the polyesters or PEAs.

Exemplary classes of therapeutic and/or prophylactic agents include, but are not limited to, anti-analgesics, anti-inflammatory drugs, antipyretics, antidepressants, antiepileptics, antipsychotic agents, neuroprotective agents, anti-proliferatives, such as anti-cancer agents (e.g., taxanes, such as paclitaxel and docetaxel; cisplatin, doxorubicin, methotrexate, etc.), anti-infectious agents, such as antibacterial agents and antifungal agents, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated. These may be vitamins, supplements such as calcium or biotin, or natural ingredients such as plant extracts or phytohormones.

The agents can be small molecules, i.e., organic, inorganic, or organometallic agents having a molecule weight less than 2000, 1500, 1200, 1000, 750, or 500 amu, biomolecules or macromolecules (e.g., having MW greater than 2000), or combinations thereof.

Examples of small molecule therapeutic agents include, but are not limited to, acyclovir, amikacin, anecortane acetate, anthracenedione, anthracycline, an azole, amphotericin B, bevacizumab, camptothecin, cefuroxime, chloramphenicol, chlorhexidine, chlorhexidine digluconate, clortrimazole, a clotrimazole cephalosporin, corticosteroids, dexamethasone, desamethazone, econazole, eftazidime, epipodophyllotoxin, fluconazole, flucytosine, fluoropyrimidines, fluoroquinolines, gatifloxacin, glycopeptides, imidazoles, itraconazole, ivermectin, ketoconazole, levofloxacin, macrolides, miconazole, miconazole nitrate, moxifloxacin, natamycin, neomycin, nystatin, ofloxacin, polyhexamethylene biguanide, prednisolone, prednisolone acetate, pegaptanib, platinum analogues, polymicin B, propamidine isethionate, pyrimidine nucleoside, ranibizumab, squalamine lactate, sulfonamides, triamcinolone, triamcinolone acetonide, triazoles, vancomycin, anti-vascular endothelial growth factor (VEGF) agents, VEGF antibodies, VEGF antibody fragments, vinca alkaloid, timolol, betaxolol, travoprost, latanoprost, bimatoprost, brimonidine, dorzolamide, acetazolamide, pilocarpine, ciprofloxacin, azithromycin, gentamycin, tobramycin, cefazolin, voriconazole, gancyclovir, cidofovir, foscarnet, diclofenac, nepafenac, ketorolac, ibuprofen, indomethacin, fluoromethalone, rimexolone, anecortave, cyclosporine, methotrexate, tacrolimus and combinations thereof.

In one embodiment, the particles contain an anti-tumor agent. Classes of antitumor agents include, but are not limited to, angiogenesis inhibitors, DNA intercalators/crosslinkers, DNA synthesis inhibitors, DNA-RNA transcription regulators, enzyme inhibitors, gene regulators, microtubule inhibitors, and other antitumor agents.

Examples of angiogenesis inhibitors include, but are not limited to, Angiostatin K1-3, DL-α-Difluoromethyl-ornithine, Endostatin, Fumagillin, Genistein, Minocycline, Staurosporine, (±)-Thalidomide, revlimid, and analogs and derivatives thereof.

Examples of DNA intercalators/cross-linkers include, but are not limited to, Bleomycin, Carboplatin, Carmustine, Chlorambucil, Cyclophosphamide, cis-Diammineplatinum(II) dichloride (Cisplatin), Melphalan, Mitoxantrone, Oxaliplatin, analogs and derivatives thereof.

Examples of DNA-RNA transcription regulators include, but are not limited to, Actinomycin D, Daunorubicin, Doxorubicin, Homoharringtonine, Idarubicin, and analogs and derivatives thereof.

Examples of enzyme inhibitors include, but are not limited to, S(+)-Camptothecin, Curcumin, (−)-Deguelin, 5,6-Dichlorobenz-imidazole 1-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, Tyrphostin AG 879, and analogs and derivatives thereof.

Examples of gene regulators include, but are not limited to, 5-Aza-2′-deoxycytidine, 5-Azacytidine, Cholecalciferol (Vitamin D3), Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, Troglitazone, and analogs and derivative thereof.

Examples of microtubule inhibitors include, but are not limited to, Colchicine, Dolastatin 15, Nocodazole, Paclitaxel, docetaxel, Podophyllotoxin, Rhizoxin, Vinblastine, Vincristine, Vinorelbine (Navelbine), and analogs and derivatives thereof.

Examples of other antitumor agents include, but are not limited to, 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, Urinary trypsin inhibitor fragment (Bikunin), and analogs and derivatives thereof.

In other embodiments, the agent is a biomolecule, such as a nucleic acid. The nucleic acid can alter, correct, or replace an endogenous nucleic acid sequence The nucleic acid is used to treat cancers, correct defects in genes in other pulmonary diseases and metabolic diseases affecting lung function, genes such as those for the treatment of Parkinson's and ALS where the genes reach the brain through nasal delivery.

Gene therapy is a technique for correcting defective genes responsible for disease development. Researchers may use one of several approaches for correcting faulty genes: A normal gene may be inserted into a nonspecific location within the genome to replace a nonfunctional gene. An abnormal gene can be swapped for a normal gene through homologous recombination. The abnormal gene can be repaired through selective reverse mutation, which returns the gene to its normal function. The regulation (the degree to which a gene is turned on or off) of a particular gene can be altered.

The nucleic acid can be a DNA, RNA, a chemically modified nucleic acid, or combinations thereof. For example, methods for increasing stability of nucleic acid half-life and resistance to enzymatic cleavage are known in the art, and can include one or more modifications or substitutions to the nucleobases, sugars, or linkages of the polynucleotide. The nucleic acid can be custom synthesized to contain properties that are tailored to fit a desired use. Common modifications include, but are not limited to use of locked nucleic acids (LNAs), unlocked nucleic acids (UNAs), morpholinos, peptide nucleic acids (PNA), phosphorothioate linkages, phosphonoacetate linkages, propyne analogs, 2′-O-methyl RNA, 5-Me-dC, 2′-5′ linked phosphodiester linage, Chimeric Linkages (Mixed phosphorothioate and phosphodiester linkages and modifications), conjugation with lipid and peptides, and combinations thereof.

In some embodiments, the nucleic acid includes internucleotide linkage modifications such as phosphate analogs having achiral and uncharged intersubunit linkages (e.g., Sterchak, E. P. et al., Organic Chem., 52:4202, (1987)), or uncharged morpholino-based polymers having achiral intersubunit linkages (see, e.g., U.S. Pat. No. 5,034,506). Some internucleotide linkage analogs include morpholidate, acetal, and polyamide-linked heterocycles. Other backbone and linkage modifications include, but are not limited to, phosphorothioates, peptide nucleic acids, tricyclo-DNA, decoy oligonucleotide, ribozymes, spiegelmers (containing L nucleic acids, an apatamer with high binding affinity), or CpG oligomers.

Phosphorothioates (or S-oligos) are a variant of normal DNA in which one of the nonbridging oxygens is replaced by a sulfur. The sulfurization of the internucleotide bond dramatically reduces the action of endo- and exonucleases including 5′ to 3′ and 3′ to 5′ DNA POL 1 exonuclease, nucleases S1 and P1, RNases, serum nucleases and snake venom phosphodiesterase. In addition, the potential for crossing the lipid bilayer increases. Because of these important improvements, phosphorothioates have found increasing application in cell regulation. Phosphorothioates are made by two principal routes: by the action of a solution of elemental sulfur in carbon disulfide on a hydrogen phosphonate, or by the more recent method of sulfurizing phosphite triesters with either tetraethylthiuram disulfide (TETD) or 3H-1,2-bensodithiol-3-one 1,1-dioxide (BDTD). The latter methods avoid the problem of elemental sulfur's insolubility in most organic solvents and the toxicity of carbon disulfide. The TETD and BDTD methods also yield higher purity phosphorothioates.

Peptide nucleic acids (PNA) are molecules in which the phosphate backbone of oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that is similar to oligonucleotides, but are achiral and neutrally charged molecules. Peptide nucleic acids are typically comprised of peptide nucleic acid monomers. The heterocyclic bases can be any of the standard bases (uracil, thymine, cytosine, adenine and guanine) or any of the modified heterocyclic bases described below. A PNA can also have one or more peptide or amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), and the like. Methods for the chemical assembly of PNAs are well known.

In some embodiments, the nucleic acid includes one or more chemically-modified heterocyclic bases including, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives, 4-acetylcytosine, 8-hydroxy-N-6-methyladenosine, aziridinylcytosine, 5-(carboxyhydroxylmethyl) uracil, 5-bromouracil, 5-carboxymethylaminomethyl-2-thiouracil, 5-carboxymethylaminomethyluracil, dihydrouracil, N6-isopentenyladenine, 1-methyladenine, 1-methylpseudouracil, 1-methyl guanine, 1-methylinosine, 2,2-dimethylguanine, 2-methyladenine, 2-methylguanine, 3-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, 2,6-diaminopurine, and 2′-modified analogs such as, but not limited to O-methyl, amino-, and fluoro-modified analogs Inhibitory RNAs modified with 2′-flouro (2′-F) pyrimidines appear to have favorable properties in vitro.

In some embodiments the nucleic acid includes one or more sugar moiety modifications, including, but are not limited to, 2′-O-aminoethoxy, 2′-O-amonioethyl (2′-OAE), 2′-O-methoxy, 2′-O-methyl, 2-guanidoethyl (2′-OGE), 2′-O,4′-C-methylene (LNA), 2′-O-(methoxyethyl) (2′-OME) and 2′-O—(N-(methyl)acetamido) (2′-OMA).

Methods of gene therapy typically rely on the introduction into the cell of a nucleic acid molecule that alters the genotype of the cell. Introduction of the nucleic acid molecule can correct, replace, or otherwise alters the endogenous gene via genetic recombination. Methods can include introduction of an entire replacement copy of a defective gene, a heterologous gene, or a small nucleic acid molecule such as an oligonucleotide. This approach typically requires delivery systems to introduce the replacement gene into the cell, such as genetically engineered viral vectors.

Methods to construct expression vectors containing genetic sequences and appropriate transcriptional and translational control elements are well known in the art. These methods include in vitro recombinant DNA techniques, synthetic techniques, and in vivo genetic recombination. Expression vectors generally contain regulatory sequences necessary elements for the translation and/or transcription of the inserted coding sequence. For example, the coding sequence is preferably operably linked to a promoter and/or enhancer to help control the expression of the desired gene product. Promoters used in biotechnology are of different types according to the intended type of control of gene expression. They can be generally divided into constitutive promoters, tissue-specific or development-stage-specific promoters, inducible promoters, and synthetic promoters.

Viral vectors include adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA.

Gene targeting via target recombination, such as homologous recombination (HR), is another strategy for gene correction. Gene correction at a target locus can be mediated by donor DNA fragments homologous to the target gene (Hu, et al., Mol. Biotech., 29:197-210 (2005); Olsen, et al., J. Gene Med., 7:1534-1544 (2005)). One method of targeted recombination includes the use of triplex-forming oligonucleotides (TFOs) which bind as third strands to homopurine/homopyrimidine sites in duplex DNA in a sequence-specific manner. Triplex forming oligonucleotides can interact with either double-stranded or single-stranded nucleic acids. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a Kd less than 10-6, 10-8, 10-10, or 10-12. Methods for targeted gene therapy using triplex-forming oligonucleotides (TFO's) and peptide nucleic acids (PNAs) are described in U.S. Published Application No. 20070219122 and their use for treating infectious diseases such as HIV are described in U.S. Published Application No. 2008050920. The triplex-forming molecules can also be tail clamp peptide nucleic acids (tcPNAs), such as those described in U.S. Published Application No. 2011/0262406.

Double duplex-forming molecules, such as a pair of pseudocomplementary oligonucleotides, can also induce recombination with a donor oligonucleotide at a chromosomal site. Use of pseudocomplementary oligonucleotides in targeted gene therapy is described in U.S. Published Application No. 2011/0262406.

K. Formulations

The polyesters or PEAs described here can be formulated for a variety of routes of administration including, but not limited to, enteral, parenteral, topical, or transmucosal. In some embodiments, the polyesters are administered parenterally. The polyesters or PEAs can be formulated as a solution, suspension, or gel.

The particles/conjugates described herein can be combined with one or more pharmaceutically acceptable carriers to prepare pharmaceutical compositions. The compositions can be administered by various routes of administration. However, in some embodiments, the particles are administered parenterally including, but not limited to, intramuscular, intraperitoneal, intravenous (IV) or subcutaneous injection. The particles can be administered locally or systemically.

In a preferred embodiment the polyesters, PEAs or particles containing the polyesters or PEAs are administered as a solution or suspension by parenteral injection. The formulation can be in the form of a suspension or emulsion. Suitable excipients include, but are not limited to, pharmaceutically acceptable diluents, preservatives, solubilizers, emulsifiers, adjuvants and/or carriers. Such compositions can include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### III. Methods of Making

A. Grafted Polymers

1. Side Chain/Grafting Synthesis

The polymers described herein contain one or more monomers functionalized with a radio-opaque agent or radio-opaque agent-containing moiety. In some embodiments, the one or more monomers are functionalized with iodine or an iodine containing moiety. In some embodiments, an iodine-containing moiety is grafted onto the polymer after polymerization. For example, iodine-containing hydroxylamine can be prepared via the nucleophilic substitution of 2-iodobenzyl bromide by N-hydroxyphthalimide in the presence of triethylamine as shown in Scheme 1.

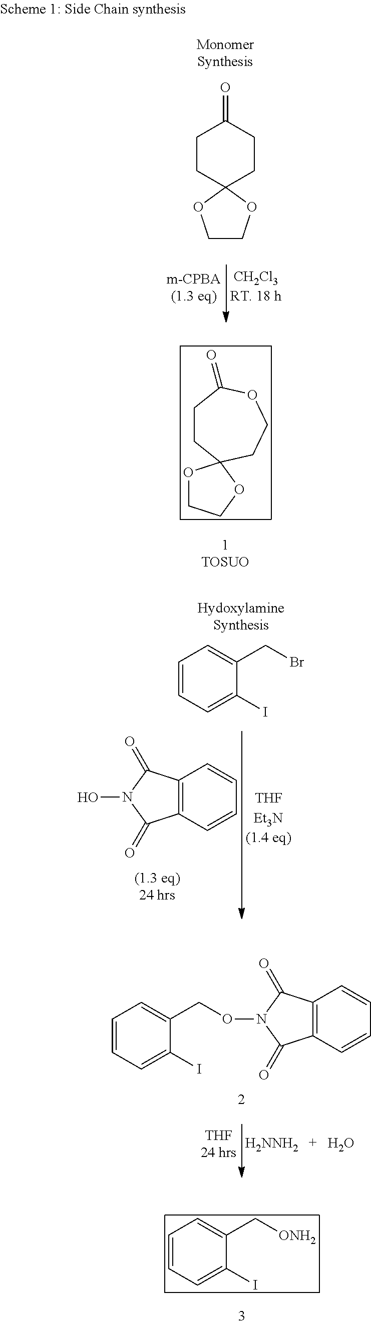

The phthalimido group can be removed by exposing the phthalimido derivative, 2, to hydrazine overnight at room temperature to yield the final product O-(2-iodobenzyl)hydroxylamine, 3, after a short column purification.

4-iodophenylalanine is a commercially available product. 4-iodophenylalanine can also be prepared using any method known in the art, such as iodination of the phenyl group of L-phenylalanine. For example, in the electrophilic iodination of L-phenylalanine, L-phenylalanine is reacted with iodine and sodium iodate, in acetic acid and sulfuric acid, followed by work up in a base to yield the 4-iodophenylalanine product.

2. Polymer Synthesis

i. Polyesters

The polymers described herein can be prepared using a variety of techniques in the art. For example, thermal ring-opening polymerization with the catalyst tin (II) octanoate can be used to prepare copolymers with a controlled incorporation of comonomer, as shown in Scheme 2.

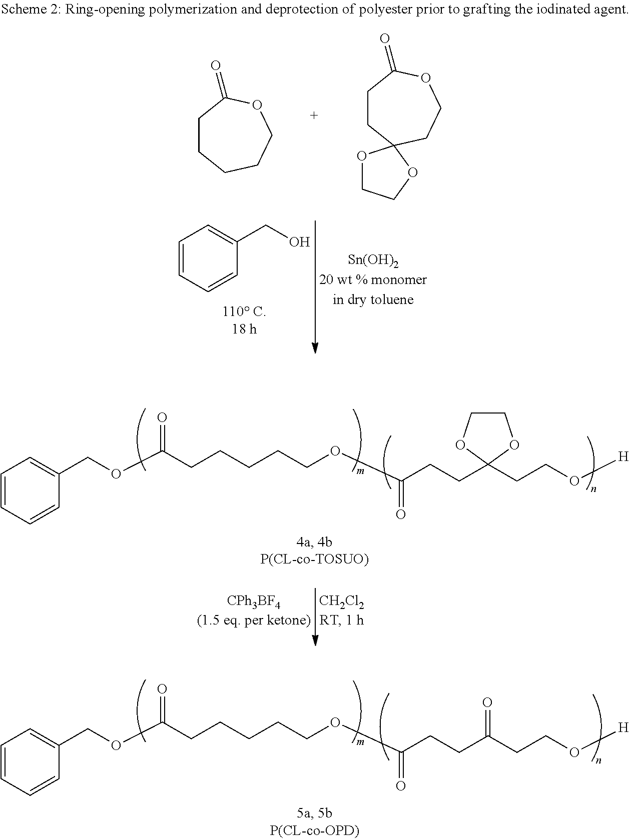

The copolymerization of ε-CL and TOSUO can be done using benzyl alcohol as the initiator in an organic solvent, such as dry toluene, at 20 wt. % monomer at 110° C. for 18 h. The reaction was monitored with ¹H NMR spectroscopy by calculating the percent conversion of monomer to polymer with the methylene unit ratios on the oxygen side of the ester with measured conversions>90% for all polymerizations. The final products were isolated from precipitation in methanol non-solvent, dried under vacuum and then characterized with ¹H NMR spectroscopy to determine a percent TOSUO incorporation and a number average molecular weight as tabulated in Table 1.

TABLE 1
Proton NMR and GPC Characterization of Copolymers and Graft Copolymers

| | CL | Functional | | | | | | |
|---|---|---|---|---|---|---|---|---|
| Polymer | Repeat | Repeat | Mole % | Mₙ, | Mₙ, | M_{w}, | | M_{w}, |
| Sample | Units^{a} | Units^{a} | Functionality | NMR^{a} | GPC^{b} | GPC^{b} | PDI^{b} | GPC^{c} |
| 4a | 21 | 3 | 12.5 | 3020 | 10800 | 17300 | 1.60 | 21600 |
| P(CL-co- | | | | | | | | |
| TOSUO) | | | | | | | | |
| 5a | 21 | 3 | 12.5 | 2890 | 13200 | 17900 | 1.36 | 22200 |
| P(CL-co- | | | | | | | | |
| OPD) | | | | | | | | |
| 6a | 21 | 3 | 12.5 | 3580 | —^{d} | —^{d} | —^{d} | 22700 |
| Graft | | | | | | | | |
| Copolymer | | | | | | | | |
| 4b | 33 | 6 | 15.4 | 4910 | 19000 | 29200 | 1.54 | 28900 |
| P(CL-co- | | | | | | | | |
| TOSUO) | | | | | | | | |
| 5b | 33 | 6 | 15.4 | 4640 | 18900 | 27800 | 1.47 | 28700 |
| P(CL-co- | | | | | | | | |
| OPD) | | | | | | | | |
| 6b | 33 | 6 | 15.4 | 6030 | —^{d} | —^{d} | —^{d} | 29800 |
| Graft | | | | | | | | |
| Copolymer | | | | | | | | |

^{a}Calculated from ¹H NMR spectra using the ratio of the —COOCH₂— methylene integrations of CL and T repeat units in the polymers and the CH₂OH methylene of the alcohol end group . These are likely underestimated values due to the sensitivity of the alcohol chain end integration on phasing and baseline correction of ¹H NMR data acquired.
^{b}GPC data acquired with a Malvern GPCMax with an RI detector and PS standards from single runs on same day.
^{c}GPC data acquired with a Waters GPC equipped with an RI detector and PS standards as the average of triplicate runs.
^{d}Data not acquired.

Subsequent removal of the ketal units can be accomplished using trityltetrafluoroborate in dichloromethane, followed by precipitation in methanol, and isolation and drying of the solid product to yield poly(caprolactone-co-1,4-oxepan-1,5-dione), abbreviated P(CL-co-OPD).

Iodinated PLA (i-PLA) (not copolymer) was generated using ring opening polymerization of 0.25 mmol of iodinated lactide (i-LA) in toluene, with 0.34 μmol of tin(II) ethylhexanoate and 0.55 μmol of several initiators for 24 hours. In this experiment the iodinated polymer was polymerized using iodinated monomer without using post-grafting methods. Other initiators of ROP that can be used are shown in Table 2, and FIG. 1c. The ROP initiators include, but are not limited to, methanol, benzyl alcohol, 2-propanol, glycerol, pentaerythitol, and PEG.

TABLE 2
Initiators of ring-opening polymerization that can be used to form polyesters

| | | | | | Reaction |
|---|---|---|---|---|---|
| Reaction | Monomer | Initiator | Solvent | Catalyst | Time |
| 2-Propanol | i-LA | 2-Propanol | Toluene | Tin(II) 2- | 24 hr |
| | (0.25 mmol) | (0.00055 mmol) | | ethylhexanoate | |
| | | | | (0.00034 mmol) | |
| Methanol | i-LA | Methanol | Toluene | Tin(II) 2- | 24 hr |
| | (0.25 mmol) | (0.00055 mmol) | | ethylhexanoate | |
| | | | | (0.00034 mmol) | |
| Benzyl | i-LA | Benzyl Alcohol | Toluene | Tin(II) 2- | 24 hr |
| Alcohol | (0.25 mmol) | (0.00055 mmol) | | ethylhexanoate | |
| | | | | (0.00034 mmol) | |
| Pentaerythitol | i-LA | Pentaerythitol | Toluene | Tin(II) 2- | 24 hr |
| | (0.25 mmol) | (0.00055 mmol) | | ethylhexanoate | |
| | | | | (0.00034 mmol) | |
| Glycerol | i-LA | Glycerol | Toluene | Tin(II) 2- | 24 hr |
| | (0.25 mmol) | (0.00055 mmol) | | ethylhexanoate | |
| | | | | (0.00034 mmol) | |
| PEG | i-LA | PEG | Toluene | Tin(II) 2- | 24 hr |
| | (0.25 mmol) | (0.00055 mmol) | | ethylhexanoate | |
| | | | | (0.00034 mmol) | |

ii. Poly(Ester Amides)

Similarly to polyesters, PEAs can be synthesized via ROP of a morpholine-2,5-dione, with tin(II) 2-ethylhexanoate (tin(II) octanoate) catalyst, as shown below in Scheme 3.

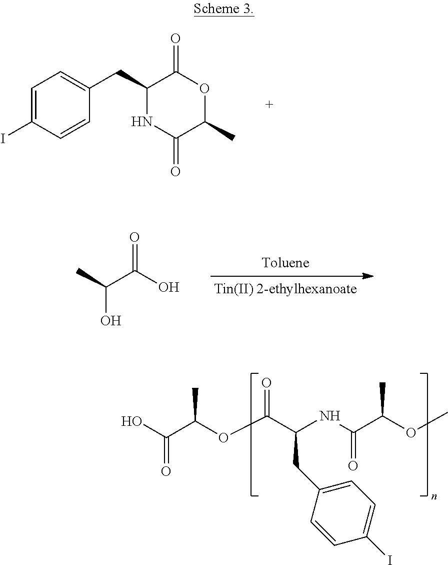

The reaction proceeds for 24 hours. In another embodiment, the ROP is performed using morpholine-2,5-dione derived from the cyclization of 4-iodophenylalanine and glycolide. The exemplified initiator of ROP is lactic acid.

The synthesis described above is representative and is in no way limiting. The polyesters or PEAs described herein can be prepared using other techniques known in the art.

3. Functionalization of Polymers

i. Polyesters

The polyesters can be functionalized using a variety of techniques known in the art. For example, attachment of O-(2-iodobenzyl)hydroxylamine to the P(CL-co-OPD) polymer backbones can be done through p-toluene sulfonic acid-catalyzed oxime formation for 24 h in THF solution, followed by precipitation into cold methanol, isolation by filtration and drying under vacuum to yield a white solid graft copolymer. To demonstrate the reproducibility and effective matching of reaction mixture and polymer product stoichiometries, each of the ketone-bearing polymers (5a and 5b) was exposed to 1.1 equivalents of hydroxylamine per ketone under the conditions listed above.

Proton NMR spectroscopy confirmed that ˜100% coupling was achieved on both samples (Products 6a and 6b) as can be seen by the complete shifting of the two different methylene subunits alpha to the ketone to new positions in the oxime product with a shift in the methylenes adjacent to the oxygen of the backbone ester groups. Additionally, new benzylic methylene resonances appear at 5.1. These NMR results provide evidence of a well-defined and controllable coupling reaction stoichiometry as observed through characterization of the final products. ¹³C NMR also indicated grafting of the hydroxylamine through the appearance of new aromatic resonances from 99-140 ppm, appearance of a pair of oxime isomer resonances at 156.3 and 157.1 ppm, as well as the disappearance of the C═O resonance at 206 ppm.

Gel permeation chromatography (GPC) and data tabulated in Table 1 of the 4a, 5a, and 6a products confirm that no unwanted degradation of the polymer backbone was observed during the 24 h exposure to the acid catalyst. Additionally, no dramatic change in the molecular weight distribution or peak molecular weight of the chromatogram for the graft copolymer was observed relative to the ketone-bearing and ketal-containing polymer precursors. These NMR and GPC results support the stability of the polymer under varying reaction conditions and the efficiency and accuracy of the polymer oxime graft reaction for creating iodinated poly(ε-caprolactone) materials.

ii. Poly(Ester Amides)

The PEAs can be synthesized using non-iodinated monomers, followed by iodination as described above, to yield iodinated PEAs.

Iodination can be carried out via the reaction of reactive groups on residues with other reactive groups containing moieties that include iodine. For example, carboxylic side chains can be derivatized with benzyl groups, amino side chains with benzyloxycarbonyl, isocyanate- and isothiocyanate-containing compounds, while unsaturated side chains and backbones can be derivatized via methods that include, but are not limited to, an ene-thiol reaction, an ene-amine reaction, an yne-thiol reaction, and a Huisgen 1,3-dipolar cycloaddition.

iii. Polymerization of Iodinated Monomers

An iodinated lactide monomer was prepared by a modified literature protocol starting from commercially available 4-iodophenylalanine First, 4-iodophenylalanine was converted to 2-hydroxy-3-(4-iodophenyl)propionic acid on treatment with sodium nitrite in aqueous sulfuric acid. This substance was then converted to the target 4-iodobenzyl lactide on treatment with 2-bromopropionyl chloride and triethylamine in anhydrous acetonitrile. Tin (II) octanoate-catalyzed ring-opening polymerization resulted in the radio-opaque poly(lactic acid) polymer.

The synthesis described above is representative and is in no way limiting. The polyesters described herein can be prepared using other techniques known in the art.

### IV. Methods of Using

The materials described herein can be used for any application where a radio-opaque material is desired or necessary. In some embodiments, the materials are used to form, whole or in part, a medical device.

Biodegradable polymeric implants and drug delivery systems formed from polyesters are commercially available or are in clinical trials. Examples include, but are not limited to, dental implants, cranio-maxilofacial implants, soft tissue sutures and staples, abdominal wall repair device, tendon and ligament reconstruction devices, fracture fixation devices, and coronary drug eluting stents. However, in vivo performance of these devices cannot always be predicted by mathematical modeling or common in vitro studies due to the complex biological environment associated with tissues and patient health. When these polyesters are implanted into patients, there is a risk of failure of the device, complications, need for replacement, or even death.

The devices described above lack the imaging properties that allow for locating the devices, monitoring changes in morphology, detecting cracks and defects, and/or quantitatively determining the degradation kinetics in situ using non-invasive imaging. Fluorescent biodegradable polymers have addressed some of these challenges, but are not applicable to deep tissue imaging, which is required for in vivo use in humans, and does not allow for monitoring of implant defects. To address the issue of deep tissue imaging of polymeric materials, radio-opaque contrast agents have been developed. The conventional approach to provide polymeric implants with x-ray contrast properties is by addition of radio-opaque fillers (salts and nanoparticles) in the matrix of the polymer. An example of this technology is ReZolve®, which is a coronary drug-eluting stent that is manufactured by REVA. This stent is composed of a degradable or resorbable tyrosine-derived polycarbonate polymer impregnated with iodine for radio-opacity to enable visualization with x-ray and fluoroscopy. However, these materials can suffer from leaking of the radio-opaque agent leading to decreased performance and reliability.

The polyesters or PEAs described herein provide x-ray contrast, even in deep tissue, and allow for identifying and quantifying cracks, defects and changes in morphology of the polymer and quantifying the degradation of the polymer in devices and implants. Referring to FIG. 3, the polyesters (i-PCL) described herein provide high x-ray contrast (3.5 times greater than PLA control and 3.3 times greater than poly(caprolactone-co-1,4-oxepan-1,5-dione) control not doped with iodine (PCL). Regarding deep tissue imaging, the control PLA could not be visualized with 2 mm of tissue covering the sample. In contrast, the polymers described were clearly visible for all tissue thickness measured (0.2-9 cm). Results show that the contrast intensity of the polymers described herein decreases as the thickness of the liver tissue increases. Despite the decrease in signal, the contrast intensity was significantly higher than the background (6.17 times higher, p<0.05) at a depth of 9 cm and demonstrated that the contrast intensity of polymers can be quantified through different thicknesses of tissue because of the high iodine content of the prepared polymer grafted with radio-opaque agent-containing moiety.

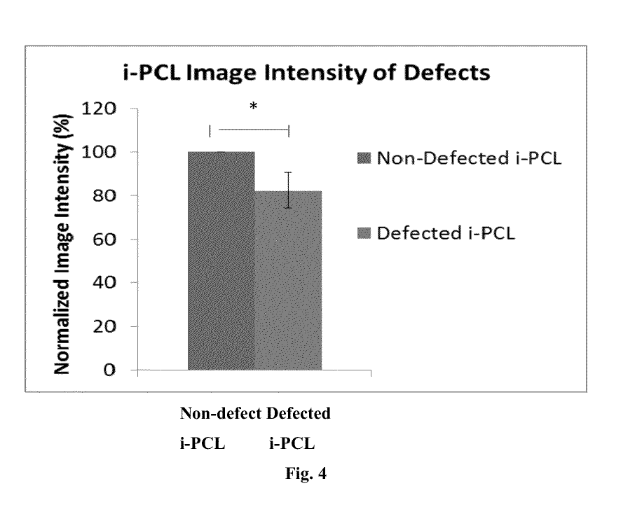

The polymers described herein were also effective as imaging contrast agents for detecting cracks and defects using x-ray imaging. Small defects were made in the polymers and were imaged using x-ray. Referring to FIG. 4, the relative x-ray image intensity of the defect samples were significantly lower than controls without defects, with the image intensity of defected samples being 18% lower than controls (n=3) (p<0.05). The defects were readily visualized through the soft tissue as well as through bone.

The materials described herein can be used to form, whole or in part, a variety of devices including, but not limited to, dental implants, breast reconstruction, cranio-maxilofacial implants, soft tissue sutures and staples, abdominal wall repair devices, scaffolds, such as tissue engineering scaffolds, tendon and ligament reconstruction devices, fracture fixation devices, skin, scar, and wrinkle repair/enhancement devices, spinal fixation and fusion devices, nanoparticles, microparticles, and coronary drug eluting stents. The materials can also be used as coatings on medical devices and implants, particularly those used subcutaneously, such as catheters; absorbable constructs for site-specific diagnostic applications; components of absorbable/disintegratable endovascular and urinogenital stents; catheters for deploying radioactive compositions for treating cancer as in the case of iodine-131 (or 123) in the treatment of prostate, lung, intestinal or ovarian cancers; dosage forms for the controlled delivery of iodide in the treatment of thyroid glands and particularly in the case of accidental exposure to radioactive iodine; components of an absorbable device or pharmaceutical product to monitor its pharmacokinetics using iodine-127, 123 or 131; and barrier film to protect surrounding tissues during brachytherapy and similar radiotherapies as in the treatment of ovarian and abdominal cancers.

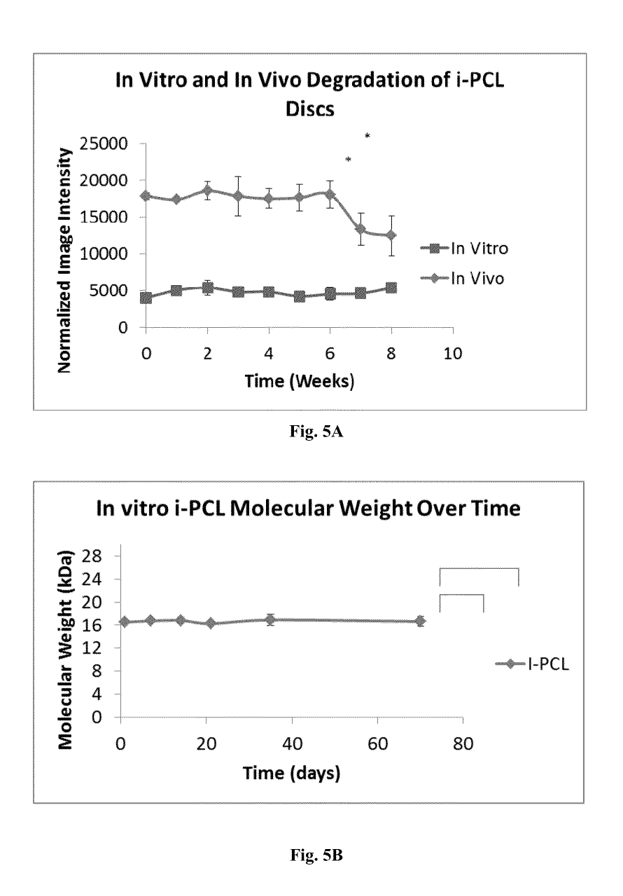

In a preferred embodiment, the devices include discs formed from i-PCL. i-PCL discs were tested for image intensity and degradation properties in vitro and in vivo over an eight-week period. Referring to FIG. 5A, surprisingly, the normalized image intensities of the i-PCL discs were significantly higher in vivo compared to their intensities determined in vitro. The intensities dropped after six weeks in vivo, which was attributed to degradation. Nonetheless, the in vivo intensities were still significantly higher than the in vitro intensities. These results show that the devices containing these polymers can be used for X-ray image analysis over at least an eight-week period. The molecular weight of i-PCL remained fairly constant in vitro in PBS over a 70-day period, FIG. 5B.

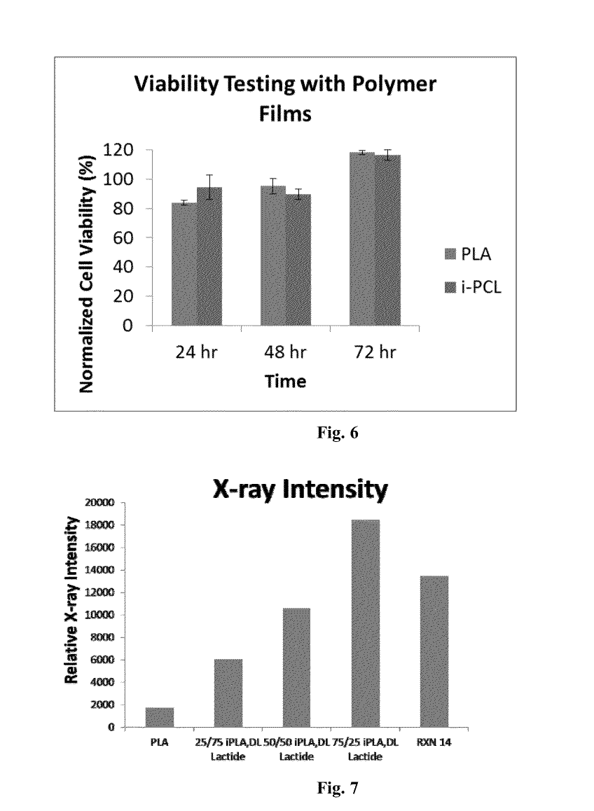

The biocompatibility of i-PCL was determined both in vitro and in vivo. The in vitro cell viability results monitored at 24 hours, 48 hours and 72 hours are shown in FIG. 6, in comparison with the known biocompatible polymer, PLA. The in vitro results show that cell viabilities were not significantly different between PLA and i-PCL, and more importantly, no adverse effects on the cells were observed during the three-day period.

For in vivo biocompatibility analysis, one PLA and one i-PCL disc were subcutaneously implanted into the back of Sprague Dawley rats (n=3), and X-ray image contrast was monitored over an eight-week period. The i-PCL discs remained visible throughout the eight-week period. Histological analysis of tissues containing i-PCL and PLA showed little immune response, as ascertained by (i) minimal cell accumulation at the implant/tissue interface in H&E stains, and (ii) a thin collagenous capsule (˜100 μm thick), which is expected to form as a provisional matrix at the site of implantation of the biomaterial.

The effects of polymer composition on X-ray image contrast intensity were also determined, FIG. 7. In some embodiments, polymers are co-polymers formed from iodinated and non-iodinated monomers. In a preferred embodiment iodinate monomer is i-LA and the non-iodinated monomer is D/L-LA. In some embodiments the i-LA/D/L-LA ratios are 0/100, 25/75, 50/50, 75/25, and 100/0, preferably the i-LA/D/L-LA ratios are 25/75, 50/50, 75/25, and 100/0, and most preferably the i-LA/D/L-LA ratio is 75/25.

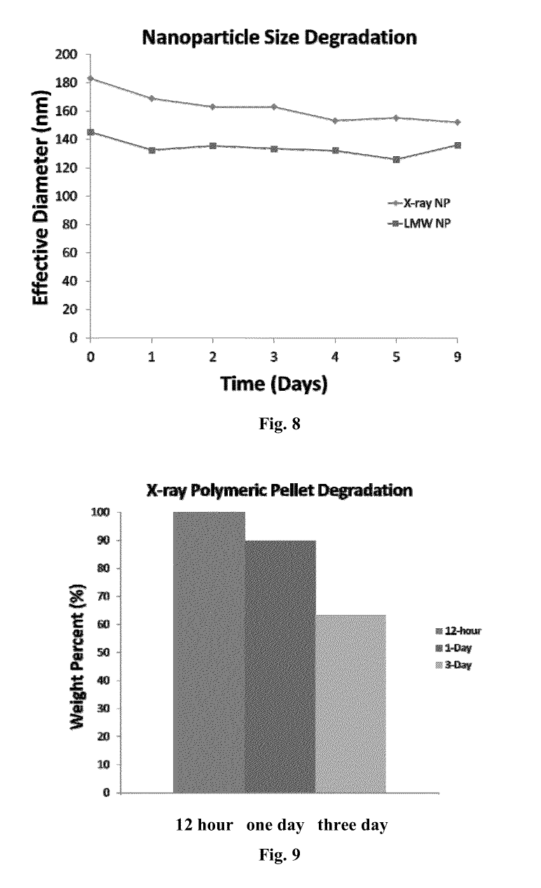

In some embodiments, the iodinated polymers are mixed with non-iodinated polymers and nanoparticles or microparticles are formed from the mixture of polymers. In some embodiments, the non-iodinated polymers are hydrophilic, hydrophobic or amphiphilic. In a preferred embodiment, iodinated polymer is i-PLA and the non-iodinated amphiphilic polymer is PLA-PEG. In a further embodiment, the ratio of PLA-PEG/i-PLA is 60/40. Referring to FIG. 8, nanoparticles formed from PLA-PEG/i-PLA in a ratio of 60/40 were fairly stable in vitro using PBS 7.4 at 37° C., as determined by the effective diameters of the nanoparticles over a nine-day period.

Referring to FIG. 9, polymeric pellet degradation monitored at 12 hours, one day and three days shows that the pellets retained about 100%, 90% and 60%, respectively of their weight. These results show that pellets formed from these polymers can be used to perform X-ray image analysis over a three-day period.

### EXAMPLES

### Materials

Tin(II) 2-ethylhexanoate ([CH₃(CH₂)₃CH(C₂H₅)CO₂]₂Sn, ˜95%), Meta-chloroperoxybenzoic acid (m-CPBA, ≦77%), 1,4-cyclohexandione monoethylene acetal (97%), 2-iodobenzyl bromide (97%), N-hydroxyphthalimide (≧97%), triethylamine (≧99%), and hydrazine monohydrate (64-65%) sodium sulfate (Na₂SO₄, >99%), anhydrous magnesium sulfate (MgSO₄, >99.5%), anhydrous toluene (C₆H₅CH₃, 99.8%), methanol (CH₃OH, >99.9%), and chloroform (CHCl₃, >99.8%) were supplied by Sigma-Aldrich.

Anhydrous sodium sulfate, sodium bisulfite, and sodium bicarbonate were purchased from Fisher Scientific and used as received.

All other solvents (ethyl acetate, hexanes, methanol (MeOH), dichloromethane (CH₂Cl₂), deuterated chloroform (CDCl₃), and tetrahydrofuran (THF)) were used as received. Toluene (Sigma Aldrich) was dried by heating at reflux over sodium and distilled under nitrogen prior to use.

D,l-lactide (C₆H₈O₄, PURASORB DL) was supplied by Purac Biomaterials.

ε-Caprolactone (CL, Sigma-Aldrich) were distilled from calcium hydride (CaH₂) and stored under nitrogen prior to use.

Para-toluenesulfonic acid monohydrate (TsOH, Sigma Aldrich) was dissolved in THF to afford a 0.02 M solution.

PrestoBlue Cell Viability Reagent was supplied by Life Technologies.

Initiators (2-propanol, methanol, benzyl alcohol, pentaerythitol, glycerol, PEG) were supplied by CL, Sigma-Aldrich.

i-D,L-lactide, 3-(4-iodobenzyl)-6-methylmorpholine-2,5-dione, 3-(4-iodobenzyl)morpholine-2,5-dione, 3-(4-iodobenzyl)-caprolactone were supplied by CL, Sigma-Aldrich.

### Instrumentation and Measurements

Proton and carbon nuclear magnetic resonance (¹H and ¹³C NMR) spectroscopy experiments were conducted using a 300 MHz Varian Mercury 300 Vx NMR spectrometer. Samples were acquired in deuterated chloroform for nt=32 or 128 for proton and nt=1024 or 4096 for carbon experiments of small molecules and polymers, respectively. Data processing and storage were achieved on a Sun Microsystem workstation. NMR figures were generated using Spinworks freeware to process the FID and then export them as text files to be subsequently plotted in overlays within Origin 7.0.

Polymer Molecular Weight (Mw) values were determined through gel permeation chromatography (GPC) on a Waters 1525 Binary HPLC pump with a Waters 2414 refractive index detector. A Waters Styragel HR 4E THF (7.8×300 (mm) ID×Length) and Shodex KF guard column were used for separation. The mobile phase was THF and polymers were prepared by dissolving in THF at a concentration of 1 mg/mL and filtering through a 0.2 μm PTFE syringe filter (VWR International). Flow rate was set at 0.8 mL/min and polystyrene standards (9, 35, 50, 100, and 200 kDa from PolySciences) were used to quantify molecular weight using a third-order fit calibration curve.

GPC data were acquired on a Malvern GPCMax equipped with an external column heater (35° C.) and Viscotek refractive index detector (VE3580) using inhibited THF as an eluent. Samples were prepared at 1.0 mg/mL in THF and filtered through 0.2 μm PTFE syringe filters (VWR International). Separation was achieved through use of the following columns in series: Malvern (CLM3008-Tcaurd) Organic Guard Column (10 mm×4.6 mm), Waters Styragel HR 4ETHF, and Malvern (T6000M) General Mixed Bed (300 mm×7.8 mm) over a 40 minute sample run with molecular weights and polydispersity calculated from a third-order calibration curve from twelve different polystyrene standards Mp ranging from 1050-3.8×106 Da.

IR spectra were recorded on Bruker Alpha FT-IR spectrometers using Opus 6.5 software.

Differential Scanning calorimetry (DSC) experiments were conducted on a Perkin Elmer DSC 7 over a range of −20 to 180° C. at 5° C. per minute. The data were then processed using the Pyris software to obtain Tm values.

Thermogravimetric Analysis (TGA) was performed on a TA Instruments Hi-Res TGA 2950 thermogravimetric analyzer by running samples from 20 to 600° C. at 10° C. per minute under nitrogen.

Explanted tissue and polymer samples were processed and sectioned via standard paraffin sectioning techniques. Samples were dehydrated using ethanol and xylene prior to being embedded in paraffin. Sections 5 μm thick were stained with hematoxylin and eosin (H&E), and Masson's Trichrome. Samples were imaged using a Nikon AZ100 multizoom microscope.

Quantitative data are presented as mean+/−standard deviation with n=3, unless otherwise indicated. Statistical analyses were performed using a two-tailed t-test and statistical significance was set at p<0.05. In figures, statistical significance is denoted by ‘*’.

### Example 1

### Synthesis of 1,4,8-Trioxaspiro[4.6]-9-undecanone (1)

1,4-cyclohexanedione monoethylene acetal (4.99 g, 32.0 mmol, 1 eq.) was dissolved in methylene chloride (50 mL) in a 300 mL round bottom flask (RBF) and was allowed to stir for 10 minutes. Meta-chloroperoxybenzoic acid (11.50 g, 48.0 mmol, 1.5 eq.) was weighed out into a 50 mL beaker and was added to the flask in scoops to the 300 mL RBF over 30 minutes. A white precipitate was noticed approximately 20 minutes after all reagents had been added. The reaction was allowed to proceed at room temperature overnight.

The contents of the reaction flask were added to a 1000 mL Erlenmeyer flask equipped with a stirbar, followed by 100 mL H₂O and 50 mL of CH₂Cl₂. Sodium bisulfate (7.67 g) was then added by scoopula to the stirring mixture over 30 minutes, followed by sodium bicarbonate (6.82 g), and allowed to stir overnight. The contents of the Erlenmeyer were then poured into a 2 L separatory funnel where the organics were collected. The aqueous layer was washed with 2×50 mL of CH₂Cl₂. The combined organic layers were then extracted with 2×50 mL of a sodium bisulfite solution, 2×50 mL with a saturated sodium bicarbonate solution and 1×100 mL of brine. The organic layer was then dried over sodium sulfate and concentrated by rotary evaporation to yield a viscous off-white oil that became a crystalline white solid under high vacuum. Yield: 4.91 g (89%)¹H NMR (300 MHz, CDCl₃, δ): 4.25 (t, 2H, —COOCH₂—), 3.94 (t, 4H, —COOCH₂CH₂—), 2.67 (t, 2H, —COCH₂—), 1.98 (t, 2H, —COOCH₂CH₂—), 1.87 (t, 2H, —COCH₂CH₂—); ¹³C NMR (75 MHz, CDCl₃, δ): 175.7 (C═O), 108.1 (ketal), 65.0, 64.6, 39.3, 32.9, 29.1 ppm.

### Example 2

### O-(2-iodobenzyl)-N-hydroxyphthalimide by nucleophilic substitution from 2-iodobenzyl bromide (2)

N-hydroxyphthalimide (2.86 g, 17.53 mmol, 1.3 eq.) was added to a 500 mL RBF using a solids funnel followed by 45 mL of THF. Triethylamine (2.8 mL, 20.1 mmol, 1.5 eq.) was added to the reaction flask using a 5 mL syringe and a red color was immediately observed upon addition. A stock solution of 2-iodobenzyl bromide (3.97 g, 13.4 mmol, 1 eq.) in THF (15 mL) was added dropwise to the reaction RBF in 3 aliquots of 5 ml. The flask was capped and the reaction was allowed to proceed at room temperature for 18 h. The crude reaction mixture was characterized with TLC using a 1:1 hexanes:ethyl acetate eluent. After removal of the THF solvent by rotatory evaporation, the reaction mixture contents were transferred into a reparatory funnel by rinsing of the RBF with methylene chloride (165 mL) and water (165 mL). After initial separation, the organic layer was set aside and the aqueous layer was extracted 2×100 mL of CH₂Cl₂. The organic layers were combined and then washed with distilled water (3×100 mL) and once with brine (100 mL). The combined organic layer was dried over anhydrous sodium sulfate in a 500 mL Erlenmeyer overnight. The organic layer was filtered the following day and concentrated by rotary evaporation and high vacuum to yield an off-white powdery solid. No further purification by column chromatography was required. Yield: 4.72 g (93% isolated). ¹H NMR (300 MHz, CDCl₃, δ): 7.92 (m, 1H, Ar H), 7.90 (m, 1H, phthalimido), 7.78 (m, 1H, phthalimido), 7.61 (d, 1H, Ar H), 7.48 (t, 1H, Ar H), 7.01 (t, 1H, Ar H), 5.35 (s, 2H, C₂H₄ICH₂—) ppm. ¹³C NMR (75 MHz, CDCl₃, δ): 163.6 (phthalimide), 139.8, 137.1, 134.7, 131.4, 130.1, 129.1, 129.7, 123.8, 99.8, 83.1 ppm; IR (solid, ATR): v=3057 (w), 2962-2854 (w), 1783 and 1723 (vs, broad over range to 1650), 1618 (w), 1607 (w), 1586 (w) 1462 (m), 1439 (m), with fingerprint peaks at 1387, 1370, 1354, 1183, 1128, 1102, 1079, 1011, 967, 875 cm⁻¹.

### Example 3

### O-(2-iodobenzyl)hydroxylamine (3)

O-(2-iodobenzyl)-N-hydroxyphthalimide (0.50 g, 1.32 mmol., 1.0 eq.) was massed into a 100 ml RBF equipped with a stirbar. To this flask, THF (15 ml) was added and the mixture was allowed to stir for 15 minutes to dissolve the starting material. Hydrazine monohydrate (0.35 mL, 7.2 mmol, 5.5 eq.) was then added by syringe to the RBF and a light yellow color change was observed. Reaction was allowed to proceed for 24 h at room temperature.

Reaction mixture (murky white) was washed twice with water, once with brine, and once with methylene chloride. The mixture was purified by column chromatography with methylene chloride as eluent (increasing polarity with methanol as needed) and concentrated by rotary evaporation to afford an off-white oil. Yield: 0.32 g (97% isolated). ¹H NMR (300 MHz, CDCl₃, δ): 7.81 (d, 1H, Ar H), 7.35-7.49 (m, 2H, Ar H), 7.0 (t, 1H, Ar H), 6.51 (broad s, 2H, —ONH₂), 4.69 (s, 2H, C₆H₄ICH₂—) ppm; ¹³C NMR (75 MHz, CDCl₃, δ) 139.9, 139.7, 129.8, 128.5, 99.1, 81.7 ppm; IR (from CDCl₃ solution): v=3309 and 3235 (m, broad), 3146 (w), 3059 (w) 2920 and 2867 (w, broad), 1584, 1563, 1464, and 1436 (m), with fingerprint peaks at 1272, 1184, 1109, 1045, 1006, 944, 900, 745, 648, 1183 cm⁻¹.

### Example 4

### Thermal Polymerization using s-CL, TOSUO, Sn(Oct)₂ and Benzyl Alcohol to Afford Poly(CL₂₁-co-TOSUO₃) (4a)

Dry ε-caprolactone (6.6 mL, 60 mmol, 90 eq.) and TOSUO (3) (1.19 g, 6.9 mmol, 10 eq.; from a 2.0M dry toluene solution) was added to a 100 mL 3-neck RBF equipped with a stirbar using dry syringes and needles. An additional 4 mL of dry toluene was added to the reaction flask under inert N₂ atmosphere, followed by distilled benzyl alcohol (70 μL, 0.66 mmol., 1.0 eq.) and tin (II) octanoate catalyst (110 μL, 0.34 mmol., 0.51 eq.). The bottom of the 100 mL 3 neck RBF was submerged in a silicone oil bath with the temperature set at 110° C. The reaction was monitored by removal of an aliquot for ¹H NMR analysis at 18 h and was subsequently quenched with 2 drops of p-toluene sulfonic acid (0.2 M in THF). The reaction mixture was precipitated in 1500 mL of cold methanol to yield white solid that was collect on a fritted funnel and dried under vacuum. Yield: 6.72 g (87% overall yield as measured from 96% conversion of ε-CL and 94% conversion of TOSUO). Confirmed final product as poly(CL₂₁-co-TOSUO₃). ¹H NMR (300 MHz, CDCl₃, δ): 7.35-7.4 (m, 5H, Ar H), 5.12 (s, 2H, benzylic H of end group), 4.15 (m, 2H, —CH₂OCO-TOSUO), 4.05 (t, 2H, —CH₂OCO-CL), 3.95 (s, 4H, —OCH₂CH₂O-TOSUO ketal), 3.65 (t, 2H, —CH₂OH end group), 2.39 (t, 2H, —OCOCH₂-TOSUO), 2.30 (t, 2H, —OCOCH₂-CL), 2.05-1.90 (m, 4H, —OCOCH₂CH₂C(OCH₂CH₂O)CH₂CH₂O-TOSUO), 1.60 (m, 4H, —OCOCH₂CH₂CH₂CH₂CH₂O-CL), 1.40 (m, 2H, —OCOCH₂CH₂CH₂CH₂CH₂O-CL) ppm. ¹³C NMR (75 MHz, CDCl₃, δ) 173.8, 173.6, 128.8, 128.4, 109.6, 77.5 (not CDCl₃) 65.3, 64.5, 64.3, 62.7, 60.5, 60.4, 36.2, 34.4, 34.3, 32.8, 32.5, 28.9, 28.8, 28.5, 25.7, 25.5, 24.9, 24.8, 24.7 ppm; T_{m, DSC}=44.7° C. (range 40.1-46.3° C.).

### Example 5

### Synthesis of polylactide (PLA) for comparative examples

Polylactide was used in the comparative examples described below. Polylactic acid (PLA) was synthesized via ring-opening polymerization using lactic acid as the initiator and tin (II) 2-ethylhexanoate as the catalyst. Briefly, lactic acid, lactide monomer, and Na₂SO₄ were vacuum-dried overnight in the reaction vessel before use. Reagents were dissolved by stirring in anhydrous toluene under N₂ gas and reflux (120° C.). Tin(II) 2-ethylhexanoate was added and the reaction vessel was stirred at 120° C. for 24 hours under N₂ and reflux. The next day, the polymer product was washed in chloroform/water, dried over MgSO₄, and precipitated in cold methanol.

### Example 6

### Polymeric Ketal Deprotection using Trityltetrafluoroborate to afford of Poly(CL₂₁-co-OPD₃) (5a)

P(CL₂₁-co-TOSUO₃) (1.98 g, 0.710 mmol., 1.0 eq. of polymer with 3.0 eq. of ketone) was transferred into a 500 mL round bottom flask followed by 200 ml, of CH₂Cl₂. Trityltetrafluoroborate (0.94 g, 12.8 mmol., 1.3 eq. per ketone) was added to the stirring flask and a bright yellow/orange color was observed. The reaction was allowed to proceed for 1 h. The reaction mixture was added by pipette into 1500 mL of ice cold methanol and allowed to stir for >3 h. The white solid product was isolated over a fritted funnel and dried with vacuum. Yield: 1.40 g. (74% isolated)

¹H NMR (300 MHz, CDCl₃, δ): 7.35-7.4 (m, 5H, Ar H end group), 5.12 (s, 2H, benzylic H), 4.35 (m, 2H, —CH₂OCO-OPD), 4.05 (t, 2H, —CH₂OCO-CL), 3.65 (t, 2H, —CH₂OH end group), 2.80-2.75 (two t, 4H, OCOCH₂CH₂COCH₂CH₂O-OPD), 2.39 (t, 2H, —OCOCH₂-OPD), 2.30 (t, 2H, —OCOCH₂-CL), 1.60 (m, 4H, —OCOCH₂CH₂CH₂CH₂CH₂O-CL), 1.40 (m, 2H, —OCOCH₂CH₂CH₂CH₂CH₂O-CL) ppm. ¹³C NMR (75 MHz, CDCl₃, δ) 206.0, 173.7, 173.5, 172.9, 128.8, 128.4, 77.5 (not CDCl₃), 64.7, 64.3, 62.7, 59.4, 59.3, 41.7, 37.6, 34.3, 34.1, 33.6, 32.5, 28.54, 28.47, 28.0, 25.72, 25.67, 25.5, 24.8 24.6 ppm; T_{m, DSC}=57.1° C. (range 55.4-58.4° C.).

### Example 7

### Oxime-grafting of O-(2-iodobenzyl)hydroxylamine onto P(CL₂₁-co-OPD₃) to afford Graft Copolymer P(CL₂₁-co-(OPD-g-(2-IBn))₃) (6a)

P(CL₂₁-co-OPD₃) polymer (0.203 g, 0.0703 mmol polymer containing 0.211 mmol ketone) was massed into a scintillation vial equipped with a stirbar and to it was added 3 mL of THF. A 10 mL stock solution of O-(2-iodobenzyl) hydroxylamine (0.10 M) was prepared in a different vial and 2.35 mL of hydroxylamine stock were subsequently delivered by syringe to the reaction vial. Three drops of a THF stock solution of TsOH (0.02 M) were added to the reaction vial and the reaction was allowed to proceed with stirring for 24 h at room temperature. The contents of the vial were then precipitated into 300 mL cold hexanes, followed by collection by filtration and drying under vacuum. Yield: 0.177 g (79% isolated) P(CL₂₁-co-(OPD-g-(2-IBn))₃)¹H NMR (300 MHz, CDCl₃, δ): 7.83-7.81 (dd, 1H, Ar H3), 7.4-7.35 (m, 5H, Ar H end group), 7.35-7.31 (dd and td, 2H, Ar H3 & H5), 6.98 (td, 1H, Ar H4), 5.12 (s, 2H, benzylic H), 5.1 (d, 2H, —CH₂ON-oxime), 4.27 (m, 2H, —CH₂OCO-oxime), 4.05 (t, 2H, —CH₂OCO-CL), 3.65 (t, 2H, —CH₂OH end group), 2.70-2.45 (three t, 6H, OCOCH₂CH₂C(oxime)CH₂CH₂O-OPD), 2.30 (t, 2H, —OCOCH₂-CL), 1.60 (m, 4H, —OCOCH₂CH₂CH₂CH₂CH₂O-CL), 1.40 (m, 2H, —OCOCH₂CH₂CH₂CH₂CH₂O-CL) ppm. ¹³C NMR (75 MHz, CDCl₃, δ) 173.8, 173.5, 172.9, 157.1, 156.3, 140.5, 139.5, 139.4, 129.52, 129.48, 128.3, 98.2, 98.1, 79.5, 64.8, 64.4, 34.3, 34.2, 34.0, 30.5, 28.6, 25.8, 24.8 ppm; T_{m, DSC}=37.4 and 42.7° C. (range 26.9-44.1° C.).

### Example 8

### Preparation of 4-iodobenzyl lactide

α-hydroxy-4-iodo-benzenepropanoic acid and triethylamine were dissolved and stirred at 0° C. under nitrogen. After 5 minutes, 2-bromopropionyl chloride was added and the solution was stirred for an additional 30 minutes at 0° C. Triethylamine was added and the reaction was stirred and refluxed at 70° C. for 3 hours. The solution was cooled to room temperature, organic phases were combined, and washed.

### Example 9

### Characterization of functionalized polymers

Given the influence of thermal stability and crystallinity on the potential in vivo degradation of the synthetic iodine-grafted PCL material, thermal analysis by differential scanning calorimetry (DSC) was performed on each of the polymer precursors and the final grafted product. As expected, both the P(CL-co-TOSUO) initial copolymer 4a and the oxime graft product 6a display lower melting transition temperatures than unfunctionalized pure PCL (Tₘ˜60° C.) while the P(CL-co-OPD) 5a melts at higher temperatures. These results are expected due to the disruption of the crystalline packing of the polymers arising from the spiroketal and bulky aromatic side chains on the P(CL-co-TOSUO) and oxime graft product, respectively, and the increased regular packing and improved crystalline structure with the intermediate ketone-bearing OPD polymer. The lower Tₘ range (35° C.>Tₘ>50° C.) for the final graft copolymers is particularly interesting since a material Tₘ near physiological temperatures could have a significant impact on the material degradation in vivo.

To learn how the compositional and structural changes of the oxime graft copolymer affect the thermal stability of the system, thermogravimetric analysis (TGA) was performed. Main chain PCL degradation and depolymerization were observed for the OPD and oxime graft copolymers at temperature≧400° C. as expected, while the starting ketal copolymers degraded as a whole at significantly lower temperatures. Moreover, a second thermal degradation mode for the P(CL-co-OPD) polymers including 5a was observed. This mode is believed to represent the β-elimination mechanism resulting from the methylenes adjacent to the ketone units, and it has been previously documented for this class of polymers and begins at temperatures as low as 150° C. Finally, the oxime graft copolymers, including 6a, also demonstrated two different decomposition modes. One mode had a rate of mass loss that peaked at ca. 325° C., with an appropriate mass scale to be the removal of the oxime/graft side chains. The other decomposition rate peaked around 425° C., in agreement with the main chain PCL degradation/depolymerization observed for PCL-type materials.

### Example 10

### X-ray contrast imaging properties of functionalized PCL

X-ray imaging was performed under the guidance of technicians at the Godley-Snell Research Center. A Tingle 325MVET x-ray machine was used with 51 kVp, 300 mA and 5 millisecond exposure time. To test whether the polymeric materials (PLA and i-PCL) could be visualized using x-ray imaging and to demonstrate potential applications, the polymers were made into different geometries and imaged.

To characterize the x-ray contrast imaging properties of the i-PCL, a series of in vitro and ex vivo experiments were conducted. Small discs (25 mg, 5 mm in diameter, 1 mm thick) were fabricated from polylactic acid (PLA), poly(caprolactone-co-1,4-oxepan-1,5-dione)-iodine polymer (PCLOD) and i-PCL and placed into a non-treated 96-well plate. Wells were filled with phosphate buffered saline (PBS) and incubated at 37° C. and 5% CO₂. Right after being placed in PBS and each week following, the plates were imaged using x-ray. Prior to the x-ray each week, the PBS was replaced with fresh PBS. The x-ray images were analyzed using ImageJ. The average image intensity of wells filled with PBS was subtracted from the wells containing the polymeric discs.

The i-PCL disc has a high x-ray contrast and the x-ray signal intensity was 3.5 times greater than the control PLA disc (p<0.05) and 3.3 times greater than the non-functionalized control poly(caprolactone-co-1,4-oxepan-1,5-dione) polymer disc (p<0.05) (see FIG. 3A). Since PLA and PCLOPD had similar contrast properties, further studies were carried out using PLA and i-PCL discs to save PCLOPD materials for i-PCL synthesis. To demonstrate the broad spectrum of potential applications, PLA and i-PCL were shaped into common polymeric devices, such as a biodegradable rectangular implant, a biodegradable staple and a biodegradable tube and imaged using x-ray.

To test the contrast properties of i-PCL in deep tissue, polymer (PLA and i-PCL) discs were covered with varying thicknesses of porcine liver and imaged by x-ray (see FIG. 3B).

Polymers (PLA and i-PCL) were fabricated into discs (25 mg each). Porcine liver was obtained from Snow Creek Meat Processing and sectioned into thin uniform slices of known thickness. The slices of liver were placed on top of the polymeric discs to simulate increases in tissue depth inside the human body. X-ray images were taken without liver and then each time after liver slices were placed on top of the polymeric discs. X-rays images were processed using ImageJ and the image intensities of just liver tissue were subtracted from the image intensities with the polymeric discs.

The PLA disc could not be visualized with x-ray imaging with 2 mm (the smallest thickness) of liver covering the material and was still not visible when liver thickness was increased (FIG. 3B). However, the i-PCL disc is clearly visible with x-ray imaging for all thicknesses of liver (0.2-9 cm), confirming contrast properties relevant to clinical applications (FIG. 3B). The x-ray image intensity of the PLA and i-PCL discs was measured using ImageJ (NIH) and normalized to the background liver tissue at each thickness (FIG. 3C). The i-PCL disc contrast intensity decreases as the thickness of the liver tissue increases. Despite the decrease in signal, the contrast intensity was significantly higher than the background (6.17 times higher, p<0.05) at a depth of 9 cm and demonstrated that the contrast intensity of i-PCL can be quantified through different thicknesses of tissue.

To evaluate the use of the novel polymer imaging contrast agent for detecting cracks and defects using x-ray imaging, small defects were made in i-PCL and were imaged by x-ray (see FIG. 4). Small defects were made in i-PCL discs and the x-ray image intensities were compared to control i-PCL discs without defects. To test the sensitivity of the imaging technique with the contrast agent in the i-PCL, the polymeric discs were placed under a rabbit and imaged with x-ray.

The relative x-ray image intensity of the defect samples were significantly lower than controls without defects, with the image intensity of defected samples being 18% lower than controls (n=3) (p<0.05) (FIG. 4). To test if the defects could be visualized through tissue, i-PCL discs were fabricated, covered by a rabbit, and imaged using x-ray. Results showed that the defects were easily identifiable through the soft tissue of the rabbit. Further, the defect was readily visualized through the bone of the rabbit. These results confirm that x-ray imaging is sensitive to changes in morphology of the i-PCL and that these changes, or defects, can be quantified and visualized through soft and hard tissues.

To assess the use of the polymer imaging contrast agent for monitoring in vitro degradation, PLA and i-PCL discs (25 mg) were placed in a 96 well plate, submerged in PBS, and imaged weekly with x-ray. The PLA discs are not visible, and the i-PCL discs are visible through the PBS, which is consistent with previous studies. The in vitro x-ray images of i-PCL suggest that the material degrades minimally over 8 weeks (see FIG. 4A), as is expected under in vitro conditions. GPC analysis (FIG. 4B) shows that the molecular weight of the polymer is 16.5 kDa, and does not change over time when submerged in PBS over 10 weeks, supporting the in vitro imaging results.

### Example 11

### X-ray contrast imaging properties of functionalized co-polymers

A. Co-Polymer of iLA and D,L-Lactide

Pellets formed from co-polymers that were synthesized using different ratios of i-LA and D,L-lactide monomers for the ring opening polymerization, were investigated for X-ray image contrast. The X-ray intensity also showed a direct dependence on the amount of iodine present, FIG. 5. For instance, for pellets formed from the following mixtures of polymers: 25%/75% iPLA/D,L-lactide, 50%/50% iPLA/D,L-lactide and 75%/25% iPLA/D,L-lactide, the relative X-ray intensities were about three, five and nine times, respectively, higher than the intensity of pellets formed from non-iodinated PLA. Surprisingly, the X-ray intensity of the pellets dropped when the pellets were formed from 100% iPLA (RXN 14), compared to the intensity at 75%/25% iPLA/D,L-lactide.

B. Mixture of PLA-PEG/iPLA

Nanoparticles were also formed from a mixture of PLA-PEG/iPLA, and were tested for image contrasting. 12 mg of PLA-PEG/iPLA were below tissues of chicken stacks of 1 cm, 2 cm, 3 cm and 4 cm, and the tissues were exposed to X-ray. Even with a mixture of polymers, the NPs in these tissues were visible at all these depths, showing that image contrasting can be achieved not only in shallow tissues, but also in deep tissues.

### Example 12

### Effects of ROP initiators on X-ray contrast imaging properties of iodinated polymers

The effects of ROP initiators on the X-ray contrast imaging properties of the polymers described herein were assessed. In a non-limiting example ROP was performed using i-LA with different ROP initiators to generate polyesters. The X-ray contrast imaging properties of the polyesters were determined. Interestingly, the PLA polymer showed a significantly lower relative X-ray intensity compared to the other initiators, FIG. 6.

Table 3 shows additional monomers from which polyesters and PEAs were synthesized via ROP. In each instance, 0.25 mmol of the corresponding monomer was polymerized, using 0.55 μmol lactic acid as initiator and 0.34 μmol tin(II) ethylhexanoate as catalyst in toluene for 24 hours.

TABLE 3
Monomers and initiators used to perform
ROP of lactide and morpholine-2,5-dione

| | | | | | Reaction |
|---|---|---|---|---|---|
| Reaction | Monomer | Initiator | Solvent | Catalyst | Time |
| i-PLA | i-LA | Lactic Acid | Toluene | Tin(II) 2- | 24 hr |
| | (0.25 mmol) | (0. 55 μmol) | | ethylhexanoate | |
| | | | | (0. 34 μmol) | |
| 50/50 i-PLA, | 50/50 i-PLA, | Lactic acid | Toluene | Tin(II) 2- | 24 hr |
| D,L Lactide | D,L Lactide | (0. 55 μmol) | | ethylhexanoate | |
| | | | | (0. 34 μmol) | |
| Morpholine | Morpholine | Lactic acid | Toluene | Tin(II) 2- | 24 hr |
| Dione no CH3 | Dione no CH3 | (0. 55 μmol) | | ethylhexanoate | |
| | (0.25 mmol) | | | (0. 34 μmol) | |
| Morpholine | Dione | Lactic acid | Toluene | Tin(II) 2- | 24 hr |
| Dione | (0.25 mmol) | (0. 55 μmol) | | ethylhexanoate | |
| | | | | (0. 34 μmol) | |

The stabilities of the NPs composed of PLA-PEG mixed with i-PLA at a weight ratio of 60/40, respectively, were investigated in vitro using PBS 7.4 at 37° C. by monitoring changes in the effective diameters of the NPs and polymeric pellet degradation as a function of days. FIG. 8A shows the effective diameters of the NPs over a nine-day period. The effective diameters of the NPs showed a slight decrease during the first day, but remained fairly constant over an additional period of eight days. A similar trend was observed with the LMW NP, i.e., a slight drop in effective diameter was observed during the first day, but the diameters remained fairly constant for another eight days.

FIG. 8B shows the X-ray polymeric pellet degradation assessed as retained weight percent as a function of days. No noticeable degradation of the X-ray polymeric pellets was observed during the first 12 hours. At the end of the first day, about 90% of the weight of the polymeric pellets was retained. Over a three-day period, the polymeric pellets retained about 60% of their weight. The observed drop in mass of the pellets, but retention of the effective diameter of the NPs may be attributed to degradation of the polymeric pellets with a loss in weight. These data show that the polymer pellets are degrading over time herein, and can be used for X-ray imaging analysis over at least a three-day period.

### Example 13

### Biocompatibility of functionalized PCL

To evaluate the effect of PLA and i-PCL on cell viability, rat aortic smooth muscle cells were seeded onto films prepared from the polymers.

Primary rat aortic smooth muscle cells were cultured in monolayer cultures using Dulbeco's Modified Eagle Medium:F-12 (ATCC, 1:1, DMEM:F-12) supplemented with 10% fetal bovine serum (Atlanta Biologics) and 1% penicillin-streptomycin-amphotericin (MediaTech, Inc.) at 37° C. and 5% of CO₂.

Polymers (PLA and i-PCL) were dissolved in acetonitrile (ACN, 50 mg mL⁻¹) and dispensed into a non-treated 96-well plate (125 μL, 6.25 mg). Plates were left overnight under a chemical hood to evaporate ACN, leaving behind a polymer film. Cells were seeded (50,000 cells per well) into well plates with no polymer films, PLA films and i-PCL films and incubated for 24, 48 and 72 hours. At each time point, a PrestoBlue cell viability assay was performed to quantify cell viability compared to polymer film free control wells with cells.

The results of a PrestoBlue viability assay showed that films made from i-PCL had no adverse effects on cell viability after 72 hours, when compared to PLA controls and no polymer film controls, and that the differences between PLA and i-PCL were not statistically significant at each time point (p>0.05) (FIG. 9). Iodine has been FDA approved as a contrast agent, it is not toxic even at high concentrations, and it is cleared rapidly through urine. Depending on the beam intensity used, the standard dose of iodine administered intravenously in humans is 400-600 mg of iodine per kilogram. Based on the weight percent data from TGA analysis, the loading of iodine is ˜20% by weight of the implant.

One PLA and one i-PCL disc (25 mg) were subcutaneously implanted into the back of Sprague Dawley rats (n=3, male, 8 weeks). One rat was not implanted with the polymer discs as a control. Immediately following implantation, and each week following, the rats were imaged using x-ray to measure the contrast intensity of the polymeric discs. The in vivo imaging results show that the i-PCL remains clearly visible throughout the duration of the study (8 weeks), while control PLA discs could not be visualized once implanted. Imaging analyses demonstrated that the relative x-ray image intensity of the i-PCL discs decreased (30%) from 17907 Da to 12691 Da after 8 weeks, suggesting that the material experienced degradation when exposed to physiological conditions in vivo. It is important to note that in weeks 7 and 8, there was significantly less image intensity, when compared to week 6 (p<0.05). After 8 weeks, the i-PCL discs were explanted for histological analysis.

The PLA discs degraded into a gel and could not be retrieved as a disc, so any remaining polymer and surrounding tissue that the disc was implanted into was explanted for analysis. Tissue from the control rat was also explanted for analysis as a control. Histological examination of the retrieved PLA and i-PCL specimens showed little immune response, as noted by minimal cell accumulation at the implant/tissue interface in the H&E stains. Further, it can be seen in the H&E stains that cells infiltrated and populated the implanted PLA and i-PCL discs including formation of blood vessels. Masson's Trichrome stain suggests that the collagen content in control and PLA samples were comparable. For the i-PCL discs, there appears to be a thin collagenous capsule (˜100 μm thick), which is expected to form as a provisional matrix at the site of implantation of the biomaterial. The results support that x-ray imaging can be utilized to measure in vivo degradation and changes in morphology of functionalized biodegradable polymers for use as biodegradable implantable devices, such as stents, staples, fibers, coatings and screws.

Overall, the concept of using a polycaprolactone-iodine radio-opaque agent and x-ray imaging to image and measure material defects and degradation has been shown to be a promising technique as it allows for a non-invasive approach for deep tissue imaging of polymeric implants. The results confirm that the functionalization of the PCL with iodine is important for imaging the polymer using x-ray imaging, when compared to PLA and PCLOD unmodified PCL. Not only can the i-PCL be imaged using x-ray, but defects and degradation can be measured in clinically relevant tissue depths, which is a critical characteristic moving forward for implantable polymeric devices in the clinic. Partnering this imaging contrast agent with x-ray imaging is expected to overcome two remaining challenges associated with imaging of polymeric materials: (1) detecting changes in polymer morphology, like cracks and defects and (2) tracking the degradation of the polymer. For degradable implants, monitoring cracks, defects and changes in morphology over time is critical to ensure that the implant is performing as desired and to detect failed implants. With permanent metallic implants, x-ray imaging is routinely used to check for structural abnormalities, misalignments and defects as the patient heals. To improve upon this, polymeric imaging agents in bioresorbable or biodegradable implants should be used to quantify cracks, defects and changes in morphology for determining whether further therapeutic intervention is required. Being able to predict the failure of implants from signal intensity over time can improve treatment options available to the patient, improve their quality of life and reduce costs associated with complications from permanent implantable devices and revision surgeries.

The results demonstrate that the i-PCL degraded, as shown by the decrease in signal intensity at weeks 7 and 8 after implantation into rats. On the other hand the in vitro study showed that the material experienced very little degradation, since the image intensity at week 8 was very similar to the initial. The image intensity of in vivo i-PCL was greater than that of the in vitro i-PCL and is most likely due to the differences in contrast for a polystyrene dish with PBS and the soft tissue of a rat. In most cases, in vivo degradation is faster than in vitro degradation. This can be attributed to the complex biological environment associated with the formation of superoxide and enzyme activity. Additionally, the location of the implant in the body will influence the degradation of polymeric materials.

It has been demonstrated that a functionalized polycaprolactone with iodine can be imaged using x-ray and that its degradation and changes in morphology can be measured over time in vivo. The studies described herein demonstrate that i-PCL can be imaged through tissue and that the image intensity can be quantified at varying thicknesses, which validates that the imaging agent is sensitive to clinically relevant tissue depths. Results demonstrated that, over 8 weeks, the relative x-ray image intensity decreased minimally in vitro, while in vivo studies showed substantial degradation in physiological conditions. Changes in image intensity of small defects in the polymer were readily detected and quantified, even while being imaged through bone. These findings suggest that functionalized polymers can be tailored to the need and application of the polymeric device (e.g. staples, polymeric nanoparticles, and drug eluting stents, etc.).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A biodegradable, radio-opaque polymer, the polymer comprising a polyester or poly(ester amide) comprising a plurality of radio-opaque agents covalently bound to the polyester or poly(ester amide) backbone.

2. The polymer of claim 1, wherein
(i) the polyester comprises one or more monomers selected from the group consisting of lactide, glycolide, caprolactone, trimethylene carbonate, p-dioxanone,1,5-dioxepan-2-one, morpholinedione, hydroxyalkanoates, aliphatic or aromatic diacid and an aliphatic or aromatic diol, two hydroxy carboxylic acids, and combinations thereof; or
(ii) the poly(ester amide) comprises one or more monomers selected from the group consisting of amino acids, morpholine-2,5-dione, diamide-diol, diester-diamide, ester-diamine, diamide-diester, acid anhydride, dicarboxylic, diol, aminoalcohol, monomers represented by Formula I 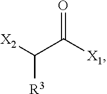
wherein X₁ is a hydroxyl group, —OR⁴, halogen, wherein the halogen is preferably chlorine; wherein R⁴ is alkyl, alkenyl, alkynyl, aryl, alkylaryl, cycloalkyl, heterocycloalkyl, heteroaryl group;
wherein X₂ is a hydroxyl group or halogen, wherein the halogen is preferably chlorine or bromine;
wherein R³ is hydrogen, or alkyl, alkenyl, alkynyl, aryl, alkylaryl, cycloalkyl, heterocycloalkyl, heteroaryl group substituted or unsubstituted with sulfhydryl, hydroxy, amino, cyano, nitro, azide, aldehyde, ester, sulfonate ester, isocyanate, thioisocyanate and carboxylic acid; monomers represented by Formula II 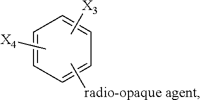
wherein the aromatic group is monoaryl, polyaryl, heteroaromatic, or combinations thereof;
wherein X₃ and X₄ are independently amine, C₁-C₁₀ amine, amide, C₁-C₁₀ amide, carboxylic acid, C₁-C₁₀ carboxylic acid, ester, C₁-C₁₀ ester, aldehyde, C₁-C₁₀ aldehyde, C₁-C₁₀ thiol, hydroxyl, C₁-C₁₀ hydroxyl, C₁-C₁₀ alkene, C₁-C₁₀ alkyne, nitro, C₁-C₁₀ nitro, cyano, C₁-C₁₀ cyano, and combinations thereof.

3. The polymer of claim 2, wherein the molecular weight of the polymer is from about 300 Daltons to about 250,000 Daltons.

4. The polymer of claim 3, wherein the degree of substitution is at least about 1%, 2%, 3%, 4%, 5%, 8%, 10%, 15%, 20%, 25%, 30%, 35%, 40%, 45%, 50%, 55%, 60%, 65%, 70%, 75%, 80%, 85%, 90%, 95%, or 99%.

5. The polymer of claim 4, wherein the degree of substitution is 100%.

6. The polymer of claim 1, wherein the radio-opaque agent is covalently bound directly to the polyester or poly(ester amide) backbone.

7. The polymer of claim 1, wherein the radio-opaque agent is covalently bound to the polyester poly(ester amide) backbone via a spacer or linker.

8. The polymer of claim 1, wherein the radio-opaque agent is iodine or an iodine-containing moiety.

9. The polymer of claim 1, wherein the polymer comprises a second polymer, wherein the polymer
(i) is a linear co-polymer of the polyester or poly(ester amide) with the second polymer,
(ii) the polyester or poly(ester amide) is mixed with the second polymer, or
(iii) the polyester or poly(ester amide) is cross-linked or inter-linked with the second polymer,
wherein the second polymer is hydrophobic, hydrophilic or amphiphilic.

10. The polymer of claim 1, wherein the polymer is an amphiphilic copolymer comprising a hydrophilic polymer and a hydrophobic polymer.

11. The polymer of claim 1, wherein the polymer is linear, branched, star-shaped, brush-shaped, comb-shaped, ladder-shaped, hyperbranched, dendrimeric polymers, or combination thereof.

12. The polymer of claim 11, wherein the cross-linked or inter-linked polymers are branched, star-shaped, brush-shaped, comb-shaped, ladder-shaped, hyperbranched, dendrimeric polymers, or combinations thereof.

13. The polymer of claim 10, wherein the hydrophilic polymer is selected from the group consisting of hydrophilic polypeptides, poly(alkylene glycols)poly(oxyethylated polyol), poly(olefinic alcohol), polyvinylpyrrolidone), poly(hydroxyalkylmethacrylamide), poly(hydroxyalkylmethacrylate), poly(saccharides), poly(hydroxy acids), poly(vinyl alcohol), and copolymers thereof.

14. The polymer of claim 10, wherein the hydrophobic polymer is selected from the group consisting of polyhydroxyacids, polyhydroxyalkanoates, polycaprolactones, poly(orthoesters); polyanhydrides, poly(phosphazenes), polycarbonates, polyamides, polyesteramides, polyesters, poly(alkylene alkylates), hydrophobic polyethers, polyurethanes, polyetheresters, polyacetals, polycyanoacrylates, polyacrylates, polymethylmethacrylates, polysiloxanes, polyketals, polyhydroxyvalerates, polyalkylene oxalates, polyalkylene succinates, and copolymers thereof.

15. The polymer of claim 10, wherein the amphiphilic polymer is PLA-PEG.

16. The polymer of claim 13, wherein the polyhydroxyacid is PLA.

17. The polymer of claim 10, wherein the hydrophilic polymer is PEG.

18. The polymer of claim 10, wherein the hydrophobic polymer is selected from the group consisting of polyhydroxyacids, polyhydroxyalkanoates, polycaprolactones, poly(orthoesters); polyanhydrides, poly(phosphazenes), polycarbonates, polyamides, polyesteramides, polyesters, poly(alkylene alkylates), hydrophobic polyethers, polyurethanes, polyetheresters, polyacetals, polycyanoacrylates, polyacrylates, polymethylmethacrylates, polysiloxanes, polyketals, polyhydroxyvalerates, polyalkylene oxalates, polyalkylene succinates, and copolymers thereof.

19. The polyester of claim 8, wherein the iodine containing moiety is selected from the group consisting of O-(2-iodobenzyl)hydroxylamine, O-(2,3,5-triiodobenzyl)hydroxylamine, (2-iodophenyl)methanethiol, (2,3,5-triiodophenyl) methanethiol, and combinations thereof.

20. The polyester of claim 19, wherein the iodine-containing moiety is O-(2-iodobenzyl)hydroxylamine.

21. The polyester of claim 8, wherein the polyester comprises iodinated lactide.

22. The poly(ester amide) of claim 8, wherein the iodine containing moiety is selected from the group consisting of O-(2-iodobenzyl)hydroxylamine, O-(2,3,5-triiodobenzyl)hydroxylamine, (2-iodophenyl)methanethiol, (2,3,5-triiodophenyl) methanethiol, i-D,L-lactide, 3-(4-iodobenzyl)-6-methylmorpholine-2,5-dione, 3-(4-iodobenzyl)morpholine-2,5-dione, 3-(4-iodobenzyl)-caprolactone, 3-iodo-1,5-dibenzoic acid, 2-iodo-4-nitrobenzoic acid, 3-iodo-4-nitrobenzoic acid, 2-iodo-4-aminobenzoic acid, 3-iodo-4-cyanobenzoic acid, 3-hydroxy-5-iodobenzoic acid, and methyl 3-amino-5-iodobenzoate, 3-amino-5-iodophenylacetic acid, methyl 2-(aminomethyl)-5-iodobenzoate, 3-formyl-4-iodobenzoic acid, 5-cyano-2-iodobenzoic acid, ethyl 3-amino-5-iodophenylacetate, 3-amino-5-iodobenzamide, 5-nitro-3-iodobenzamide and combinations thereof.

23. The poly(ester amide) of claim 22, wherein the iodine containing moiety is selected from the group consisting of i-D,L-lactide, 3-(4-iodobenzyl)-6-methylmorpholine-2,5-dione, 3-(4-iodobenzyl)morpholine-2,5-dione and 3-(4-iodobenzyl)-caprolactone.

24. Medical devices selected from the group consisting of nanoparticles, microparticles, and medical implants comprising or having coated thereon or therein the polymer of claim 1.

25. The devices of claim 24, further comprising one or more therapeutic or prophylactic agents.

26. The devices of claim 24, wherein the average diameter of the particles is from about 2 nm to 50 microns.

27. The device of claim 24 in a pharmaceutically acceptable carriers.

28. The device of claim 24 wherein the implant is selected from the group consisting of dental implants, breast reconstruction implants or meshes, cranio-maxilofacial implants, sutures, pins, screws, staples, abdominal wall repair devices, tissue engineering scaffolds, tendon and ligament reconstruction devices, fracture fixation devices, skin, scar, and wrinkle repair/enhancement devices, spinal fixation and fusion devices, stents, implantable catheters, catheters for deploying radioactive compositions, and barrier film to protect surrounding tissues during brachytherapy.

29. A method of making the biodegradable, radio-opaque polymer of claim 1, the method comprising functionalizing one or more monomers with a radio-opaque agent or a radio-opaque agent-containing moiety and polymerizing the one or more monomers to form the polymers.

30. A method of making the biodegradable, radio-opaque polymer of claim 1, the method comprising polymerizing one or more monomers to form the polymer and grafting onto the polymer a plurality of radio-opaque agents or radio-opaque agent-containing moiety.

31. A method for imaging an implantable medical device, the method comprising implanting or injecting the device of claim 24, and imaging the device.

32. The method of claim 31, wherein the device is imaged by x-ray.

33. The method of claim 31, wherein the device can be imaged through deep tissue.

34. The method of claim 31, wherein cracks or defects in the device can be imaged.

35. The method of claim 31, wherein the degree of degradation can be quantified.

36. The method of claim 31, wherein the polymer is administered as a solution, suspension, or gel.
